(19) United States
(12) Patent Application Publication
(10) Pub. No.: US 2009/0216140 A1
(43) Pub. Date: Aug. 27, 2009

(54) DEVICE AND METHOD FOR THE ELECTRICAL MEASUREMENT OF BODY FUNCTIONS AND CONDITIONS

(76) Inventor: Falko Skrabal, Graz (AT)

Correspondence Address: Workman Nydegger;1000 Eagle Gate Tower, 60 East South Temple, Salt Lake City, UT, 84111, US

(21) Appl. No.: 12/090,861

(22) PCT Filed: Oct. 23, 2006
(86) PCT No.: PCT/AT2006/000431

§ 371 Date: Oct. 23, 2008

(30) Foreign Application Priority Data
Oct. 21, 2005 (AT) A 1724/2005

(51) Int. Cl. A61B 5/0402; A61B 5/04; A61B 5/053

(52) U.S. Cl. 600/509; 600/547

(57) ABSTRACT

At an electric instrument used for the measurement of heart- and vascular function as well as body spaces, which is based on impedance measurement, the position of the electrodes, body volume and the volume of the segments located between the electrodes are measured with the help of a contactless measuring apparatus. The instrument comprises a plurality of electrodes capable of being attached to a body, a reference surface on which the body may be placed, and an electrical impedance meter capable of measuring the bodily functions of the body by measuring the electrical impedance and estimating the shape of the body by locating the electrodes attached to the body with reference to the reference surface, wherein the measured electrical impedance is associated with the estimated shape of the body segment.

Since one century the electrical activity of the heart has been recorded in millions of patients yearly in the form of an ECG. In the present patent application it is demonstrated how without appreciable additional work and expense and within the same time span with the same staff a complete recording of the mechanical activity of the heart (function), of arterial und venous perfusion of the extremities, an image of the body composition, quantification of the fluid in individual compartments (spaces) and therefore a very precise image of body homeostasis can at the same time be obtained using a very low-priced apparatus. If this is possible routinely without substantial additional costs of time and money, this new function & spaces—ECG will replace the former purely electrical ECG.

One of the unsolved main problems in the care of ill, especially critically ill people is the assessment of the fluid status, of body composition and of hemodynamic support. (“The art of fluid administration and hemodynamic support is one of the most challenging aspects of treating critically ill patients” Citation from E. W. Ely & G. R. Bernard in: Transfusions in critically ill patients, editorial NEJM 340:467-468, 1999). There are no convenient methods to assess routinely e.g. dehydration, over hydration, loss of extracellular volume, intrathoracic fluid content, ascites, intracellular volume etc in order to navigate therapy. Therefore physicians are still relying on very old and little reliable signs like turgor of the skin, the tone of ocular bulbus, etc; also ultrasound methods with measuring the diameter of the vena cava are little reliable and in many instances it will be necessary to insert a catheter into the vena cava for the measurement of central venous pressure. Also, this invasive measurement of central venous pressure is little reliable for assessing the necessity of fluid administration especially in persons suffering from heart conditions. Other methods like simple impedance measurements have also proved a failure for the management of critically ill people. So a recent Medline search in July 2005 using the terms impedance, extracellular fluid volume and intensive care with more than a thousand respective citations for the single terms did not give a single joint hit and impedance measurements are not routinely used at intensive care units. The estimation of “fat free mass”, respectively of body water and body fat with the help of impedance measurements has so far required the consideration of weight, height, age and sex, which are used in the equations for estimation of these parameters. The purpose of including these estimation parameters is to estimate the absolute volume of the body in order to be then able to “estimate” the partial volumes. With intensive care patients or patients with heart and kidney diseases it is not sufficient to estimate and it is necessary to detect the partial volumes with the accuracy of at least one litre or, if possible, even more accurately. Irrespective, it is often impossible and difficult to measure height and weight in these patients, since these patients are bed ridden. Besides, the measured variables determined with the aid of impedance deviate from each other by up to 10% in the same patients with repeated measurements. An attempt to assess changes in fluid status in acutely ill surgical patients on the basis of multi-frequency impedance measurements showed namely that the method gives significant results for a group of patients but the method was unsuited for individual patients since negative and positive correlations between actual fluid changes and impedance results were found (Chiolero R L et al. Intensive Care Med. 1992: 18 (6) 322-6). Another study showed that changes in fluid balance could be recognised by bioimpedance only if weight differences were more than 3 kg (and therefore litres) !! (Roos A N et al Critical Care Med. 1993, Jun. 21 (6), 871-7).

In medicine there is also often the necessity for the measurement of the mechanical action of the heart. So different processes and devices such as echocardiography with or without colour Doppler are used to measure the force of contraction, inotropy, and contractility and ejection fraction. Furthermore the amount of blood which is extruded from the heart with a single heart beat, the stroke volume and other hemodynamic parameters are often measured. From this and the heart frequency cardiac output can be calculated. From the mentioned parameters the function of the heart can be derived, the diagnosis of heart diseases can be made and new physiological knowledge can be gained. However, the monitoring of patients with severe heart disease at intensive care units or during anaesthesia with echocardiography is not practicable, because an examiner would have to be present continuously. Because of the importance of the problem there are multiple other methods for the measurement of cardiac output (CO) in medicine. So e.g. a catheter is inserted into the pulmonary artery and/or aorta and with the help of an indicator substance which could be warmth, cold, saline or lithium, the cardiac output is measured according to the Fick' principle from the decay of this concentration of the named indicator substance. The disadvantage of this method is the insertion of a catheter into a human blood vessel with all resulting complications such as bleeding and infection.

In recent times it has been attempted to use the Fick principle also for the measurement of cardiac output through the measurement of the concentration of gases in the exhaled air. This is possible because a rapid exchange of gas between blood and breathing air occurs, so that the concentration in both media is virtually the same. If a gas is mixed to the breathing air, the concentration of it rises also in the blood, if the administration of gas is ended, the concentration of this gas in the blood and also in the exhaled air falls, so that from the decay of the concentration of the gas over time cardiac output can be measured again according to the Fick principle. One method which has been shown to be especially advantageous is the CO2 re-breathing whereby a loop is placed into the airways of the patient and the patient breathes for a certain time his own exhaled air again, so that the CO2 concentration in blood rises. The disadvantage of these methods is that the patient must be equipped with a mouth piece and the breathing must be as constant as possible in order to achieve a uniform concentration of gas in the breathing air and in the blood. Therefore this method and equipment is mainly used during general anaesthesia with constant breathing volume and constant breathing frequency. A further process and equipment uses a similar method whereby instead of CO2 a mixture of inert gases is inhaled, which equilibrates rapidly with the blood and which is used for the measurement of cardiac output.

Another process and equipment is the measurement of stroke volume and of other hemodynamic parameters from the pulse wave which is recorded at a peripheral artery. A change of pulse wave form is caused also by changes in stroke volume and of other hemodynamic parameters, so that changes of stroke volume and other hemodynamic parameters can be estimated indirectly from a transfer function. This method has to be calibrated once initially with one of the above described processes and equipments, the method is also not accurate enough. Another method is the transcutaneous measurement of an indicator substance e.g. “indigo green” at the capillaries of the ear or the finger, which reduces the accuracy of Fick principle markedly.

Another method is the impedance cardiography (ICG). With this method a constant alternating current field is applied to the thorax and the change of alternating voltage, which arises through this alternating current field, indicates a change of fluid content in the thorax. To be more precise the alternating current resistance (impedance) is measured with this method, which is a measure of the change in the thoracic fluid content. The change in thoracic fluid content is a measure for the volume of blood expelled which each heart beat. From stroke volume and other hemodynamic parameters (SV) and from heart rate (HR) cardiac output can be calculated (CO=SV×HR). The main problems of impedance cardiography how it is still used today are manifold: In order to be able to interpret the change of fluid volume in the thorax with the heartbeat, first the true fluid content in the thorax would have to be known, which has been achieved with impedance measurements very poorly in the past. Furthermore it has so far been little known which fluid shifts, namely fluid displacement into the aorta, into the lung artery, shifts of blood within the lung vessels etc cause the change of the impedance signal with the heartbeat.

Usually for the ICG a pair of electrodes which lead a current into the body are placed on the upper and lower thorax aperture. Within this pair of electrodes a further pair of electrodes is placed for the measurement of the resulting alternating voltage. The distance between electrodes therefore is dependent on the length of the thorax and it will be described in the following as the electrode distance. So far circular electrodes or spot electrodes similar to ECG electrodes have been used for this purpose. In the patent application “Medizinische Elektrode” (ⁱ) a new alignment of electrodes has been described whereby over a short distance on the same membrane two parallel band electrodes are placed, the distance of which is accurately and reproducible given by the common supporting membrane. One of these parallel band electrodes which are placed on the common membrane is used for the application of the measuring current, the other parallel band electrode is provided for the dissipation of the measuring voltage. The upper pair of electrodes can be placed e.g. on the neck, the lower electrode pairs on the left and right side of the lower thorax aperture. This placement of electrodes shows better reproducibility of results as compared to previously used circular electrodes and also better than the spot electrodes described in the patent application U.S. Pat. No. 4,450,527 SRAMEK (ⁱⁱ). ⁱFORTIN J, NESSLER B, NESSLER W, SKRABAL F: “Medizinische Elektrode”, A 392/2001, KL. A61B, filed on 13 Mar. 2001, EP 1377 212 B1ⁱⁱSRAMEK B: “Noninvasive Continuous Cardiac Output—Anzeige-Monitor” U.S. Pat. No. 4,450,527, May 22, 1984

Disadvantages of the described impedance procedures and equipments were that the results were calculated according to KUBICEK formula (ⁱⁱⁱ,^{iv}) ore SRAMEK formula (^{v},^{vi}) respectively, which both were derived with markedly simplifying assumptions about the human body. In the former the electrode distance as measured on the body surface, in the latter body height is used. These assumptions are only correct to a limited extent, therefore a considerable error occurs in the calculation of stroke volume, of other hemodynamic parameters and of cardiac output. First and foremost in heart disease with low stroke volume, the inclusion of weight and height in the formula overestimates stroke volume because bias toward normal values will be produced (^{vii}). In recent times the impedance method has been “improved” further by using not only height but also weight within the formula for the estimation of stroke volume. This in other words means that the result is guessed mainly through the size of the body. Indeed, this trick helps to achieve an acceptable agreement with “gold standard” methods like the Fick principle in healthy subjects, because cardiac output of a healthy body fits to the size of the body like a very closely tailored suit. But who is interested in the cardiac output of a healthy subject, which can be estimated very accurately from weight, height, sex and age (as measure for metabolically active body mass) without any measurements (^{viii}). With deviation of the cardiac output from normal values the process is simultaneously less useful because in heart failure not anymore height and weight determine heart function but the number of diseased heart muscle cells. In the patent application PCT AD 03/00302 a new process and instrument for impedance cardiography has been described, whereby with the measurement at different places and with different frequencies an improvement of impedance cardiography could be reached. Especially, in this procedure the essentially same segment of the body is measured at two slightly different lengths. Particularly an “operative length” could be calculated which improved the prediction of stroke volume and ejection fraction as measured with “gold standard” methods. This method was named multisite-frequency electromechano-cardiography (_{msf}ELMC). A further improvement of this method is that no assumptions about the human geometry must be made and no models are built but, instead, in a “black box approach”, only those electrically measured parameters are used in the prediction equation which have been shown in a partial regression analysis to contribute to the prediction in a statistically significant and clinically relevant manner. ⁱⁱⁱKUBICEK, W. G., I. N. KARNEGIS, R. P. PATTERSON, D. A. WITSOE, R. H. MATTSON: Development and evaluation of an impedance cardiac output system. Aerospace Medicine 37, 1208-1212 (1966)^{iv}KUBICEK, W. G., F. J. KOTTE, M. U. RAMOS, R. P. PATTERSON, D. A. WITSOE, J. W. LA BREE, W. REMOLE, T. E. LAYMAN, H. SCHOENING, D. SMITH: The Minnesota impedance cardiograph—theory and applications. Biomed. Eng., 9, 410-416, (1974)2^{v}SRAMEK, B: Noninvasive technique for measurement of cardiac output by means of electrical impedance. Proceedings of the Vth ICEBI Tokyo, (1981)^{vi}SRAMEK, B. BO, D. M. ROSE, A. MIYAMOTO: Stroke volume equation with a linear base impedance model and its accuracy, as compared to thermodilution and magnetic flow meter techniques in humans and animals. Proceedings of the VIth ICEBI, Zadar, Yugoslavia, S. 38 (1983)^{vii}Skrabal F et al. Europ J Heart Failure Multisite Frequency Electromechanocardiography for the prediction of ejection fraction and stroke volume in heart failure 7: 974-83, 2005^{viii}Cohn J N et al. Hypertension 26: 503-508, 1995

In the present application a new method for the measurement of heart function and of body spaces, the so called function and spaces (FS)—ECG is described. Particularly for the present application, the following patent applications were considered and delineation against them is made. In U.S. Pat. No. 6,015,393 (Hovland) a method for the measurement of penal tumenescence is presented in which a measurement of the total length of the penis and a small segment of the penis is made separately, but in this patent application the total penis length is not measured at two slightly different measuring distances. In U.S. Pat. No. 6,339,722 (Heethaar) the impedance of the thorax is measured a second time at a different place of the thorax in order to obtain the allocation of intracellular water/extracellular water. In the application DE 10249863 A1 (Beise) it is attempted to measure continuous blood pressure by the ICG and by the measurement of the pulse wave. A similar approach is also described in EP 1344489A1 (Medero) and in EP 1344489 (Medero) whereby in the former a pulse-oximeter, in the latter an impedance measurement at a place different from the thorax is used in order to obtain a continuous blood pressure measurement from the change of pulse wave velocity. In the patent application U.S. Pat. No. 5,642,734 (Rubens) and in U.S. Pat. No. 5,526,808, the same body segment of the finger is measured at two different frequencies and the hematocrit is calculated therefrom. Multiple electrodes, which are introduced into the body, are also described in U.S. Pat. No. 5,109,870 (Silny) for the measurement of peristalsis of the bowel and in U.S. Pat. No. 4,951,682 (Petre) for the measurement of cardiac output with introduction of the electrodes into the heart. A training of a neuronal net for the calculation of CO is described in U.S. Pat. No. 6,186,955 (Baura), whereby this net must contain a training set as an integrative component for training on the individual patient and must obviously contain also a gold standard measurement as a reference. This is obviously needed because the net cannot generalize for every individual patient so that it is valid for the individual patient.

In the present application a system for the measurement of fluid and fluid shifts within the body and its segments is described, which allows a complete und automatic analysis of body compartments, of hemodynamics, of fluid shifts within the body without the presence of a physician and without a learning phase on the individual patient. It is amongst other things intended to combine this process and equipment e.g. with a multi channel ECG in order to obtain in the same operation procedure without loss of time or without any further expense a complete fluid and hemodynamic analysis with the help of the function and spaces (FS)—ECG. The physician receives a report which diagnoses not only the electrical but also homeostatic and hemodynamic disturbances. In the analysis can be contained among others: muscle mass, fat mass, extra cellular volume, intra cellular water, degree of leg. oedema, fluid accumulation in the abdomen (ascites), fluid accumulation in the thorax (lung oedema), extracellular and intrathoracic fluid volume, pleural effusions with side localization, arterial perfusion disturbances of the legs with localization of the side, thrombosis of the veins or venous insufficiency with side localization, furthermore ejection fraction (EF), stroke volume (SV), CO, heart failure class NYHA I-IV, estimated level of natriuretic peptide (e.g. PRO-BNP), furthermore preload and afterload, vascular elasticity, compliance of large and small vessels, augmentation index, blood pressure, peripheral resistance, baroreceptor reflex sensitivity, autonomic nervous system tone, preload of the heart, cardiovascular prognosis like the probability of cardiac events, furthermore hematocrit, serum sodium concentration, etc.

This instrument could then be named function and spaces (FS)—ECG because it for the first time comprises besides electrical function of the heart also its most important function namely the maintenance of body homeostasis and the function as a pulsatile pump. “Most important function” is in so far the right term, as the electrical function of the heart would be less interesting as long as the mechanical function of the heart is optimal for the respective situation. So far over one century one had to content oneself with the assessment of the electrical function of the heart because the mechanical action of the heart could not be assessed with the same procedure.

This instrument shall naturally also be used on intermediate and intensive care units for the continuous assessment of the patient, for the management of patients on cardiovascular drugs and for the fluid management, and it will also be of advantage at least with part of the components in each general practice.

In order to achieve acceptance by the physician and the patient, multiple prerequisites must be fulfilled:
1. If the examination must be executed fast without efforts, it would ideally be performed in the same procedure with a habitually and routinely used method like the ECG. This appears necessary, because working time of the medical staff represents the greatest cost factor in medicine and some valuable methods do not find entrance into the routine because additional staff would be needed. For the practicability the number of used electrodes should be reduced to a minimum.
2. The information of the method should be of great practical medical interest and despite the simple procedure, it should be very precise and readily reproducible in order to be used in every day life.
In the described application it is possible with only 7 to 9 double respectively triple electrodes (less than for an ECG) simultaneously with a contactless measurement of body volume and the volume of its segments to obtain precisely the above parameters.

According to the invention, these advantages are thus achieved in that, during the assessment of electric parameters, simultaneously and unnoticed a contactless three dimensional image of the examined body is produced, from which the actual volume of the total body and simultaneously of its segments can be calculated. Simultaneously, also the exact localisation of the electrodes is obtained by the contactless measuring instrument so that the electrode distance and the cross sectional area of any arbitrary sliver of the body segment lying between the electrodes can be obtained and herewith also the exact volume of the segment lying in between. Thereby, simultaneously, not only e.g. the extracellular and intracellular water could be measured electrically much more accurately, but also absolutely in litres and in percent of body volume, which is the best international standard. Simultaneously the distribution of tissues of all body segments can be assessed.

The instruments for the measurement of surfaces and of volumes could be e.g. distance measurement instruments or perhaps in addition angle measurement instruments like they are known from current techniques and like they can be bought cheaply from serial production. These instruments could be radio-ultrasound or optical measuring instruments, which in a known manner show physical properties of the body like the volume (which is obtained from the distance of the surface of the body to the measuring instrument or the measuring instruments, respectively also from the distance of body parts in relation to the distance of the examination table). Simultaneously also the exact position of the electrical electrodes placed on the body respectively of other sensors and also the distances between the individual electrodes and sensors from each other can be obtained. For that purpose the reflected or scattered signal could be analysed. Usually for that purpose interferometry, time of flight or a triangulation method are used. For the triangulation method infrared light is particularly useful which could give the angle and therefore the distance to the object e.g. with the help of a CCD or CMOS. In the most simple case a digital image of the patient in more than one plane e.g. with the help of so-called charge coupled devices (CCD) or CMOS would be sufficient in order to calculate the volume of the body and of its segments with special algorithms.

Also the (e.g. white light) phase measurement profilometry (PMP) technique could be used. In order not to disturb the equipment through ambient light an infrared projector together with cheap digital cameras could be used.

Meanwhile also cheap processes are available like the photogrammometry to produce from digital pictures a three dimensional picture of the body and thereby to calculate its surface and volume (Applied Physiol online). The technique is also described in detail under the title “Stereo photographic Digital Topography” from Mikat R. P. (www.css.edu/users/tboone2/asep/mikat.doc). This method can e.g. also be combined with the reference point technique, where a pattern of lines respectively points is projected onto the body from which distortions the three dimensional picture can be reconstructed accurately. A good overview over other older possible processes is described in Herron R E Biostereometric measurement of body form, Year book of Anthropometry 16:80-121, 1972. Of course, also the use of other methods upcoming in the future for the obtainment of surface respectively of volume of body segments is envisioned. It could e.g. be advantageous to picture a known measuring rod together with the examined body in order to correct for the varying distances and angles between the contactless surface measurement instruments and the body. These varying distances and angles arise mainly from the use of a mobile measuring station, which, in each case, is moved to the examined patient.

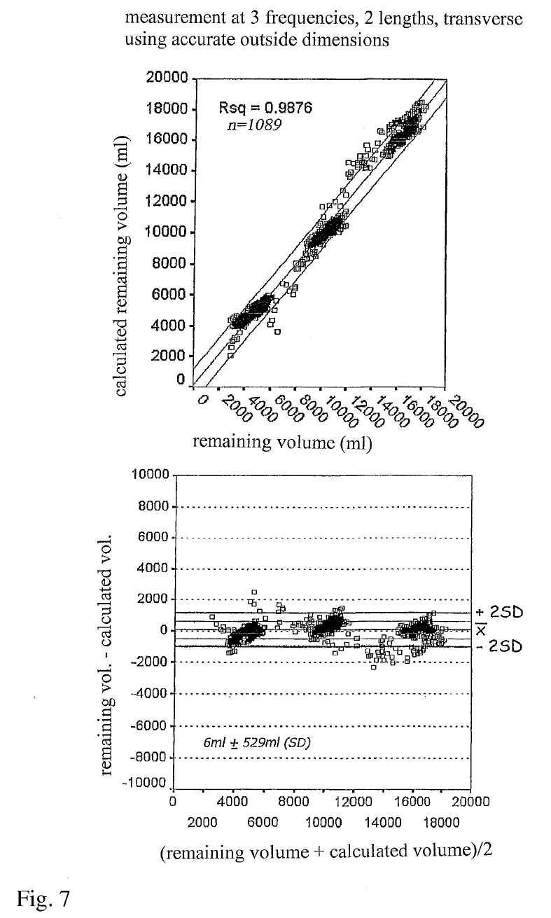

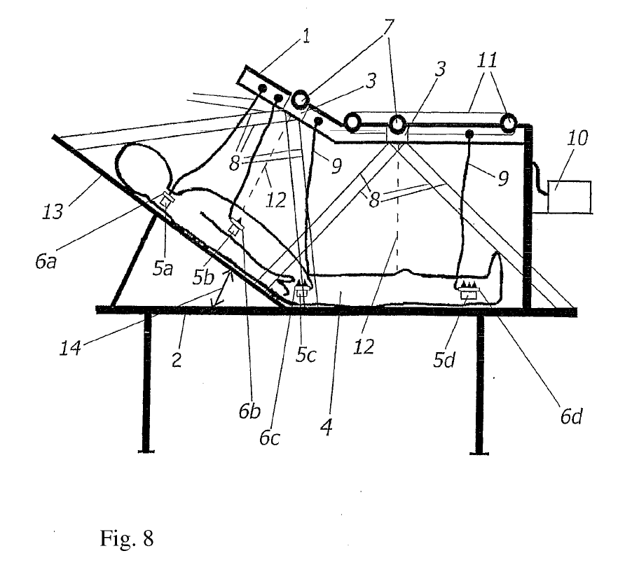

As another useable contactless process e.g. the displacement method is named, where e.g. the examined body is placed in a closed chamber and the displacement, respectively according to Boyle's law the compression of the displaced air can be measured. This alternative method is not so advantageous among other things because it is not easy to obtain the volume of different of body parts separately. With the described contactless processes it is simultaneously also possible to assess the correct position of the electrodes, while the electrodes or their electrode brackets can be recognised by the contactless surface measurement instruments by their characteristic profile or other physical properties like reflection, colour or emission of e.g. electromagnetic or optical waves, exactly in the range in which the used sensor equipment is sensitive. By the knowledge of distance and angle of the different electrodes to the measuring arm the direct distance between the electrodes can be obtained at once. It is important to recognise the real distance between the electrodes because only the knowledge about the true distance, respectively the true volume lying between the electrodes enables to recognize a possibly disturbed hydration in this body segment (see FIG. 7 and FIG. 8). Especially advantageous for the recognition of electrode position are electrode attachments which on the basis of special physical properties (e.g. profile, colour, specific reflection, oscillation, temperature etc) can be identified as an unmistakable target of the distance- and angle-measurement equipment or the digital picture. Thereby also a displaced or transposed electrode can be recognised immediately and can be signalled to the user, if each of the electrodes carries identifying information. Also with the distance measuring instruments the patient lying on the examination table can be calibrated precisely in three dimensions. With the help of the three dimensional picture of the patient the volumes of the different segments can be obtained very easily and the disturbed hydration and the fat content in the different compartments can be obtained for the first time accurately enough for clinical diagnostics. According to the present invention, these advantages are furthermore achieved by measuring the impedance and the change of impedance over time of different approximately cylindrical or oval body segments with a relatively constant cross sectional area in more than one direction, and this either sequentially or simultaneously at different downstream body segments. The measurement of different downstream segments has the advantage that the velocity of the volume wave can be recognised from which it can be deduced whether and how much the reflected volume wave contributes to the volume change in the thorax during the action of the heart. So far nobody has appreciated that the change of volume in the thorax (measured e.g. as dz or dz/dt of the thoracic impedance z) not only arises from the action of the heart but also from the reflection of the volume wave at the blood vessels in the periphery.

In more than one direction means that in addition to the hitherto used length direction the respective body segment is also measured transversally and/or diagonally while advantageously the principle of the four point measurement is maintained. For the measurement of the cylinder segments in the transverse diameter, indeed a true four point measurement with current electrodes lying outside of the voltage electrodes is not possible, but the respective current electrode can lie beside the voltage electrode. Also a two point measurement with execution of the respective electrode as a current and voltage electrode is envisioned while the resistance and therefore the fluid content of the skin can also be measured.

For the impedance measurement in length direction the application of the current will be usually outside of the voltage measurement points so e.g. at the head or near the head on the one hand and on the lower thoracic aperture, or on the abdomen, or on the leg, or on the foot, or near the foot on the other hand. The points for measuring the voltage could divide the body e.g. into a thoracic segment, an abdominal segment and in at least one leg segment or more than one leg segment, if applicable at least one arm segment or multiple arm segments could also be measured, in which case the current would have to be applied distally at the hand or in the vicinity of the hand. This has long been known per se as a segmental impedance measurement.

Furthermore it is proposed that the left and the right leg, also the left and the right arm respectively be measured separately in order to recognise also arterial and/or venous perfusion disturbances at the extremities.

The measurement not only in the length but also in the transversal and/or the diagonal direction has the following advantages: in order to perform accurate volume measurement, the electrically participating diameter of the cylinder (or ellipsoid or anomalously formed area) on the one hand, but also the electrically participating length of the cylindrical or oval segment must be known. Applying the patent application PCT/AT 03/00302, in which the impedance is measured over the length of the cylinder at two very similar segment lengths, it is possible to derive an operative length: This represents a virtual length, which is shortened by electrical bulges or lengthened by electrical waists, the virtual length corresponds herewith to a common measure of length (L)/cross-sectional area (A). The cross sectional area A alone cannot be derived. In contrast, the impedance measurement across the area of the circular or oval segment allows that the electrically participating area A can be derived accurately. This is more attractive than to estimate the cross sectional area with the help of measuring the circumference of that segment and using the formula ΔV=(C²L/(4π·z₀)) as suggested by Kubicek^{ix}. ^{ix}Kubicek W G et al “The Minnesota Impedance Cardiograph—Theory and Applications” Biomed Engin Sep. 9, 1974, pp 410-417

This formula works mainly for the estimation of a change of volume but not for the derivation of the absolute volume.

With the knowledge of the cross sectional area A and of L/A the true “electrically participating” dimensions of the cylinder or of the ovaloid are known, so that also the relative changes of volume can be derived very accurately from the measurement of the change of impedance dz/dt in the length and transversal directions. In order to be able to use the four point method for the transversal measurement, namely to maintain also here the separation of current and voltage electrodes, it is e.g. proposed to design the electrodes which are used for the voltage measurement in the length direction as double electrodes. In the length direction both parts of the double electrode (e.g. band or spot electrodes) could then be used e.g. as voltage electrodes, in order to calculate e.g. the operative length as suggested in patent application PCT/AT03/00302. However, for the transversal measurement, one of the two parts of the double electrode could then be used as current electrode, the other as voltage electrode, as this is described precisely later in the figures. Another advantage is that the transversal measurement is particularly suited to recognise respiratory activity, namely inspiration and expiration. The recognition of inspiration and expiration is also important, among other things, since the stroke volume changes with inspiration and expiration and since stroke volume changes can be used in intensive care medicine to assess the fluid requirements of the organism (“fluid responsiveness”). Similarly, also separate current electrodes and voltage electrodes could of course be used for the transversal measurement, which would increase the expenses and handling of the electrodes.

Although its sounds very complicated to measure all body segments not only in the length but also in the transversal or even diagonal direction in practice this is easily possible with only seven electrodes, whereby these electrodes partly or completely can at the same time be used also as ECG electrodes. Therefore the effort in effect is not greater as for the ECG alone. This is one of the relevant differences of the method from impedance tomography. Of course it is within the scope of the patent application to increase the accuracy of the method further by replacing the minimum of electrodes by a greater number of electrodes, whereby the method loses of course on elegance.

A calibration of the suggested impedance method with a gold standard method e.g. DXA, echocardiography, MRI other methods is easily possible.

Also a measurement of the examined segment in the diagonal direction is easily possible and can provide additional valuable information, without having to apply further electrodes at the body. This can be used e.g. to detect one or two sided pleural effusions within the thorax, furthermore the extracellular lung water which is especially relevant in intensive care medicine. Here the separate measurement on either side of the thorax with the help of the double electrodes placed there and with the application of the current further cranially (neck or head) on the one hand and further caudally (somewhere caudally from the thorax) on the other hand proves to be particularly useful.

It is proposed to perform the above measurements at more than one or multiple frequencies or even to perform a complete frequency sweep. Especially at least two, better four frequencies are of interest: a) at least two frequencies which penetrate the extracellular space in a frequency range between 0.1 and about 40 kHz (e.g. 0.5 and 1 to 5 kHz) and b) at least two further frequencies which penetrate also the cell membrane and thus estimate total body water in the frequency ranged between more than 40 kHz and 2 MHz, more precisely e.g. between 200 and 400 kHz. The reason to choose multiple, but at least two frequencies for each of the above body compartments is as follows: The distribution of current in the organism is very inhomogeneous due to the shifting composition of the tissues. In the vicinity of non-conveying or ill conveying structures the density of field lines increases due to their reduced interaction, because in boundary zones there is less interaction of field lines. This effect is the more pronounced the higher the frequency of the alternate current. With the measurement of two voltages for the respective compartment, the inhomogeneities of one compartment (extracellular space, e.g. at 0.5 and 1-5 kHz) and of the other compartment (total body water e.g. at 200 and 400 kHz) can be better recognised. The consequence of measuring at multiple frequencies for only one compartment and the consequence of a highly precise measurement of the outer dimensions of a compartment will be demonstrated later on the basis of phantom experiments. Also a separate measurement of resistance and reactance and the estimation of the phase angle is envisioned since these parameters give, among other things, a good insight into cell mass and functional integrity of cell mass.

The importance of an accurate gauging of compartments especially in critically ill patients has many causes. Although it sounds trivial it has to be considered that many patients on intensive care units cannot be weighed. This makes the fluid management very problematic particularly in the face of not controllable insensible losses through breathing, evaporation through the skin and losses of fluid e.g. through the stool which is very difficult to assess. Also accurate balance studies with measurement of all excretions over skin, breathing, urine and stool do not correspond to measured body weight (e.g. Roos A N, Critical Care Medicine 21, 871-77, 1993). Especially the distribution of body water to the extracellular and intracellular compartment is, of course, important, because therapy is adjusted accordingly. If intra and extracellular water and their deficits are known, the missing amount or excess of sodium and chloride (for the extracellular space) and the missing amount and excess of potassium and phosphate (for the intracellular space) can be evaluated accurately and automatically especially if serum sodium is considered. This helps the physician with substitution therapy enormously. In combination with an ECG there are also for the first time critical advantages in recognising a reduced perfusion of the heart muscle. During ischemia (reduced perfusion of the heart muscle) the myocardium becomes stiffer, the pumping performance decreases and temporary congestion of fluid in the lung follows. If, as hitherto, an ECG is performed in a patient with unclear heart complaints in order to recognise changes in re-polarization (which regrettably often are missing), an (often only intermittent) increase in intra-thoracic volume resulting from reduced heart performance can now be recognised for the first time. Thereby the ECG will become much more sensitive for the detection of ischemia. With the use of a fixed electrode distance between two electrodes also the resistance between those electrodes and thus skin perfusion and fluid content of the skin can be calculated, which also simplifies fluid therapy. With the simultaneous use of a constant current measurement (e.g. with a Wheatstone Bridge circuit or a similar circuit) particularly the fluid content of the superficial layers of the cornum strateum can be measured, with alternate current measurement the fluid content of superficial and deep layers and with capacity measurements the fluid content of the deep layers of the cornum strateum (Triebskorn A et al Dematologia 1983, 167:64-9). For the measurement of skin resistance also gel covered ECG electrodes could be used, also here multiple frequency measurement can bring advantages (Janitzki A S, Vedder N, Multichannel measurement of skin resistance. Biomed Tech 1987, May; 32 (5): 98-107). The measurement of the fluid content of the skin additionally gives important information about fluid balance of humans. It is further proposed to use in addition parameters of vascular function especially arterial function and venous function, respectively also parameters for the congestions in body segments for the calculation of heart function because vascular function influences heart function decisively. Thereby also the derived impedance curve is distorted in an unpredictable manner, if arterial function is not considered mathematically. As a parameter for the correction of the measured heart function e.g. the blood pressure, pulse wave velocity, compliance of large and small arteries and also the augmentation index^{x} has to be considered. Especially important is the continuous consideration of central aortic pressure which can be calculated accurately from the pulse wave in the radial artery. From birth to death the heart never “sees” the pressure in the brachial artery or radial artery, which is measured in medicine, but always only the pressure in the central aorta. Although this determines the afterload of the heart and although this has been known for many years this parameter is not routinely measured because additional staff would be required, which is not available. So far this has been time consuming because a mechanical transducer had to be adjusted accurately by hand above the artery. In combination with fluid coupling (Patent A 391.262, Skrabal) as described by Skrabal merely a wrist cuff needed to be fitted to the wrist without any adjustment and the central aortic pressure can be recorded routinely with every FS—ECG. This is also of advantage for the detection of ischemia because vasoconstrictive substances produced during ischemia change central aortic pressure. In addition it has been shown in invasive investigations that with fast pulse wave velocities the pulse wave arrives very early in the periphery and will so be reflected very early to the central aorta, thus resulting in the arrival of the reflection wave with an open aortic valve, which directly increases the after load to the heart. What has not been considered so far is that the volume wave of course also appears very rapidly in the periphery and also backward, which may cause an augmentation of the volume wave in the thorax and therefore also a false amplification of dz/dt. However, with the recording of thorax impedance alone only the increase of volume within the thorax during the mechanical heart action is clocked and therefore heart performance like stroke volume or ejection fraction or other parameters are falsely estimated. ^{x}Rourke M F and Gallagher D E. J Hypert 14 (suppl5) S147-157, 1996

If after load is increased due to an unfavourable vessel function this has direct consequences for volume change and therefore conductance or impedance in the thorax. An early reflection of the pulse wave, respectively volume wave back in the thorax e.g. would falsify the dz. A part of dz originates therefore not from the action of the heart, but from the reflection of the pulse wave in the periphery. This is a further factor for the previous lack of precision of impedance cardiography. So it has been demonstrated e.g. that the replacement of the central aorta with its windkessel function by a rigid glass tube immediately leads to the reduction of ejection fraction^{xi}. If conventional methods for the measurement of heart performance like ejection fraction (EF) or stroke volume are used (measured by echocardiography, isotopic methods, CT, magnetic resonance or the Fick principle) where actual EF or actual stroke volume is measured and not estimated, the result of the measurement will not be influenced by changes of afterload and the correct result will always be obtained. In contrast, with impedance cardiography changes in the afterload or in the state of the vessels will falsify the result into the opposite direction. An increase of the afterload causes then a stronger and faster volume increase in the thorax from which, in case of an increase, an improved heart performance is deduced with the current impedance cardiography, whereby the opposite is the case. So patients with heart failure show a falsified faster and greater change of impedance with the heartbeat as a consequence of possible earlier reflection of the pulse wave which results in a “falsified” higher stroke volume or falsified higher ejection fraction. The compliance of the aorta and of the great vessels as well as pulse wave velocity however can be estimated very well by simple means like pulse wave analysis, whereby it is referred e.g. to the method of Watt and Burrus^{xii}. Also the distal compliance of the small vessels has an influence on heart function since with poor compliance of these vessels the reflection zone is shifted proximally, so that an early reflection and an augmentation of the aortic pressure results, which also changes the impedance curve. Also the distal compliance can be estimated very well with the method of Watt and Burrus. In contrast to Watt and Burrus or also in contrast to the work of Cohn and Finkelstein cardiac output need not to be guessed but can be measured accurately at the same time which improves the method considerably. A further method for the measurement of augmentation of aortic pressure caused by changed arterial function is represented by the measurement of the augmentation index^{xiii}, which is also ascertained from the pulse wave. ^{xi}Urschel C W & Braunwald E: Am J Physiol 214: 298-304, 1969^{xii}Watt T B and Burrus C, J Appl Physiol 40, 171-176, 1976xiii

For the measurement of pulse wave velocity in a known manner a pressure sensitive sensor/on a distal artery, or an impedance measurement at a distal body segment, or a plethysmographic method on a distal body segment, or a transcutaneous measurement of blood gases like pulse oximetry or a Doppler flow measurement etc. can be used.

For the analysis of the shape of the pulse wave any pressure sensor fixed above the artery can be used, e.g. also a sensor with a fluid filled bladder as described in U.S. Pat. No. 6,669,648. The latter is advantageous because a very good analysis of the shape of the pulse wave is possible without positioning through medical staff.

The measurement of second impedance curve (dz or dz/dt) at a second location afar from the heart can also be used and has many further advantages. First, from this the pulse wave velocity or the change of volume velocity (independent of the actual fluid transport) can be determined as a measure for the falsification of the impedance curve at the thorax. Furthermore, from the change of the shape of dz or dz/dt in the periphery as opposed to the dz or dz/dt at the thorax further conclusions about heart function, especially stroke volume and ejection fraction, NYHA class, BNP level etc. can be drawn, so these parameters can be estimated much better as from the measurement of the impedance change at the thorax alone.

A further advantage arises if the following observation is applied: It is extremely difficult to judge the fluid needs of intensive care patients. Pulmonary wedge pressure, PWP, has proved poorly for that purpose. In contrast it has been shown that the variation of systolic blood pressure or of blood pressure amplitude with breathing is a very good measure for the fluid needs of the organism. This is especially so in patients on artificial ventilation. If, during artificial ventilation, there is little variation of systolic blood pressure or blood pressure amplitude (less than about 10%) further fluid administration does not result in an increase of ejection performance of the heart, therefore it is without sense and dangerous. This is referred to in intensive care medicine as fluid responsiveness. So by the accurate detection of breathing (e.g. through transversal measurement of impedance at the thorax as described above) on the one hand breathing can be automatically detected, on the other hand a variation of ejection performance of the heart with breathing can be obtained from impedance measurement dz/dt on a leg segment. To measure at the leg has the advantage that there, in contrast to the thorax, the dz/dt is only influenced by the heart action and not as on the thorax by its fluid content with breathing, changes in lung perfusion, air content, diameter etc. So, for the first time, without mechanical transducers and therefore artefact free, parenteral fluid requirements of severely ill patients can be recognised. Naturally also the pulse wave, e.g. with the help of fluid coupling (U.S. Pat. No. 6,669,648) or the vascular unloading technique (see application U.S. Pat. No. 6,669,648) can also be used in a conventional manner. A further advantage is that by a separate measurement of the left or right leg respectively, a one sided arterial or venous perfusion disturbance can be recognised automatically, because differences in the perfusion result also in different volume changes with the heart action. For the determination of venous perfusion disturbances it could also be advantageous to fix a tourniquet on the respective extremities and to intermittently increase or decrease the pressure in order to determine even better the venous outflow in a known manner with the help of the venous occlusion plethysmography.

However, neither the measurement of pulse wave velocity nor of volume migration velocity, nor pulse oximetry can determine the real transport of blood from one place to the other. But it is the change in circulation time, especially a slowing of blood transport which is characteristic for heart failure. This slowing is only recognisable if the transport of a bolus of blood from place A to place B can actually be measured. This corresponds to the Fick' principle. In the present application it is proposed optionally to additionally measure a bolus of changed conductance of the blood, recognisable electrically from the body surface, in one or more than one different body segments. For that purpose e.g. a substance must be brought into the circulation, which changes the conductance of the blood momentarily. This could e.g. be an injection of electrolyte solution being hypertonic or hypotonic in relation to plasma, especially a sodium chloride solution or even an isotonic electrolyte solution. This bolus produces e.g. first in the thorax or in a thoracic segment a transitional impedance change and with delay also in the following segments e.g. in another downstream thoracic segment or in the abdomen, or in the legs also an impedance change. From the impedance change in one segment, which is preferably short, and from the delay of the impedance ramp from one segment to the segment downstream important conclusions about the function of the circulation can be drawn and also EF and SV can be calculated. Thereby also the knowledge of the position of the electrodes through the contactless distance measurement as described above is of help, because the covered distance of the bolus can be measured. Thereto also the equations, how they are known from the Fick principle, e.g. from thermo-dilution can be used. An isotonic electrolyte solution is usable, because the isotonic solution which contains no formed elements like red blood cells also causes an impedance change as compared to full blood which can be measured, especially if more than one frequency is used therefor, because the conductivity of blood at different frequencies is very different depending on as to whether the erythrocyte membrane is penetrated by the alternating current or not.

A prerequisite for the method is that it functions automatically as fast as possible and unnoticed by the user. The duration of examination should ideally not be much longer than for a conventional ECG. For it several technical prerequisites are advantageous: In order to ascertain as few as possible switchovers of the applied current, it is advantageous to introduce the current in a way that different body segments can be measured with the same introduced current. The as constant as possible application of the current has the advantage that repeatedly new current fields need not be established, which would be the case with varying applications. An application e.g. near the upper end of the body, e.g. head, neck or neck region on the one hand, and the lower end of the legs on the other hand is suitable. So the thorax optionally also individual thoracic segments, the trunk and the legs can be measured simultaneously. In many cases a separate measurement of the arms need not be performed, since these represent only approx. 7% of the body volume. Body volumes can therefore be extrapolated with the inclusion of the arms. Even if an error of e.g. 10% occurred, this would falsify the final result only by 0.7%. On the other hand arms can be additionally easily examined since with the ECG arm electrodes are applied anyway. It is naturally also thought alternatively to apply the current separately for each segment and to measure the voltage. In order to measure the trunk in two segments and the legs together and/or separately, the instrument for the voltage measurement can be either multiple or the same voltage measuring instrument is used for the measurement of multiple body segments. Since nowadays a value can be determined within milliseconds and since sampling of the impedance signal is not so time critical and need not be performed with such a high time resolution, the measurement can be switched between different body segments. This is what could be measured simultaneously without change in the application of current: the thoracic segment longitudinally, or optionally diagonally in the length direction, the abdominal segment in the length direction, optionally diagonally, and both leg segments combined and/or individually.

Additionally it can be of advantage to produce templates of the impedance curves from different heart beats in order to detect a change of the signal with additionally recorded heart actions and to detect the time point when no relevant change of the impedance curve occurs anymore. At this time point, eventually already after approx. five to twenty heart beats, the impedance measurement can be ended. This template can then be analysed accurately in regard to steepness of slope, maxima, minima, partial areas and steepness of down slopes. Templates can be produced also for single phases of the circulation, e.g. for inspiration and expiration separately, because e.g. fluid responsiveness and other parameters could thus be tested. Also a selection of templates according to other criteria like e.g. minimal and maximal heart frequency can be of help.

Subsequently or simultaneously, essentially the same body segments could be measured a second time with slightly changed measuring length as is known from the patent application PCT/AT03/00302 in order to obtain a joint measure of length/area of the respective segment.

Subsequently the application of the current could be changed to a direction transversal to the length direction in order to measure the body segments also transversally. Since this transversal current application serves primarily for the volume determination of the measured body segment, the extraction of a template is unnecessary and only basal impedance is determined, so that the transversal measurement is finished within a few seconds. If the transversal measurement is used for the determination of inspiration and expiration, the measurement can take longer to produce advantageously a template during the inspiration and another template during expiration. So e.g. fluid responsiveness can be recognised as described above. The total measurement procedure can therefore be ended within minutes and thus takes no longer than the eventually simultaneous recording of the ECG. When connecting all electrodes attached to the body to the measuring apparatus (function & spaces ECG=FS—ECG) it is understood that the patient has to be protected from too high voltages or too large currents in a known manner, as is usual for all instruments which are in electrical contact with the patient. Especially, the instrument should, of course, be produced in a manner that a malfunction of the instrument, or a cardioversion of the patient e.g. in ventricular fibrillation or atrial fibrillation does not produce any damage to the patient, to the investigator or to the instrument despite the FS—ECG being connected. This is anyway current technology and is implemented in various ways in all instruments which are in clinical use.

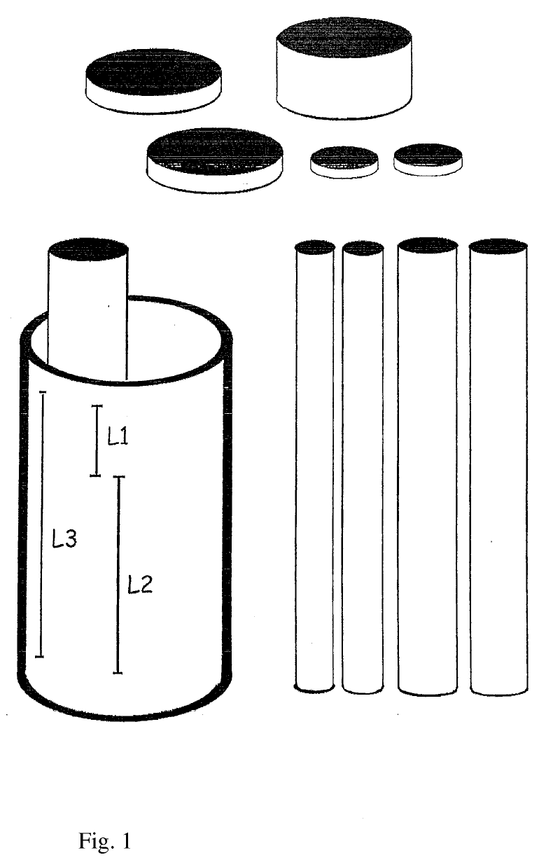

The instrument and the advantages of the method are described subsequently in FIG. 1 to FIG. 18, so is shown:

FIG. 1: A cylinder phantom with extreme variation of fluid content.

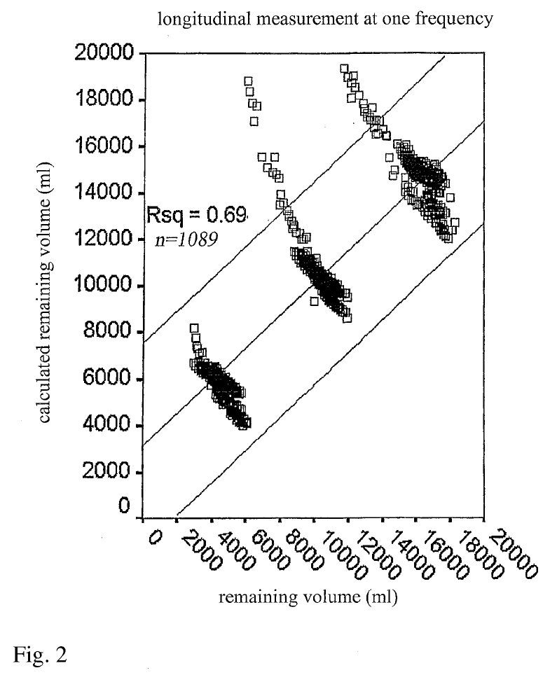

FIG. 2: Estimation of the volume within the phantom with the help of impedance measurement using one frequency.

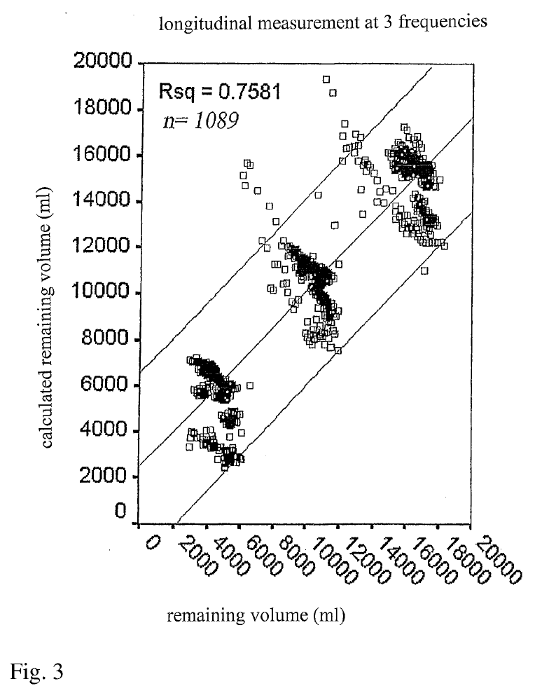

FIG. 3: The measurement of the phantom with multiple frequencies.

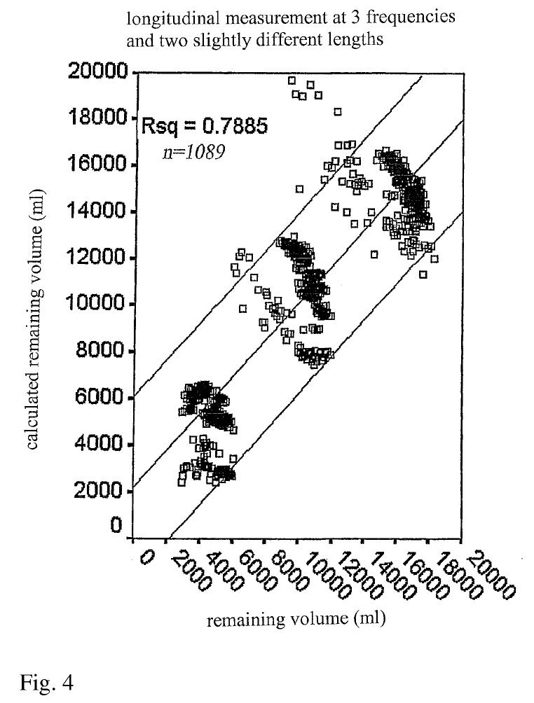

FIG. 4: The measurement of the phantom with multiple frequencies and variable length.

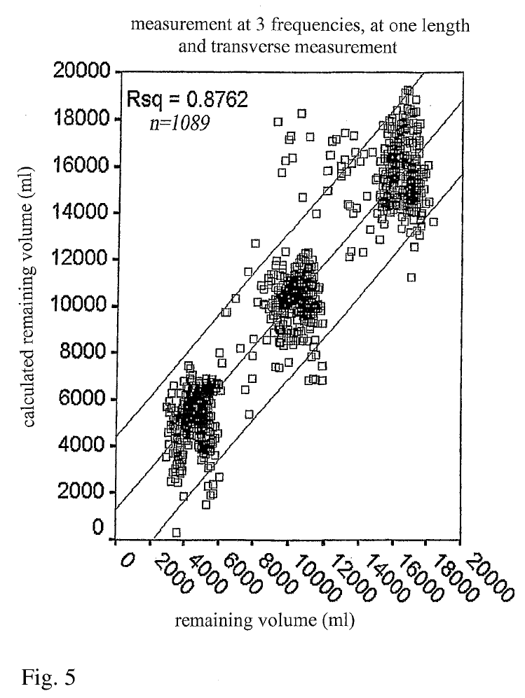

FIG. 5: The measurement of the phantom with multiple frequencies and length and transversal measurement.

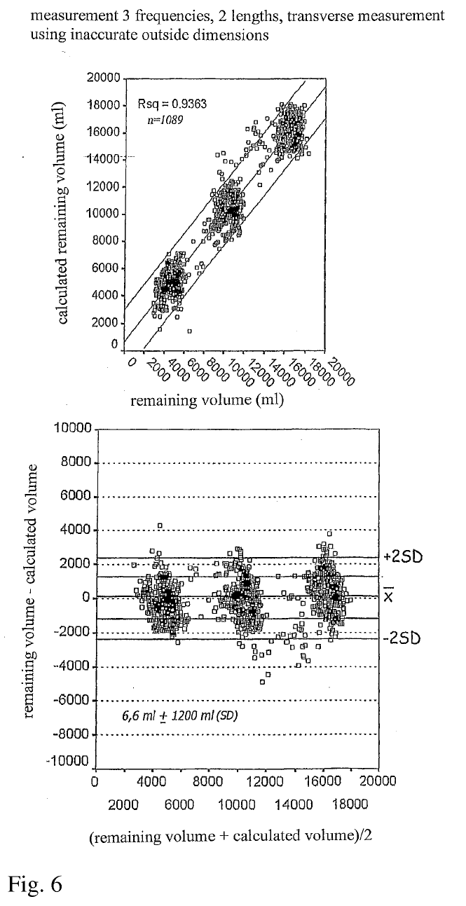

FIG. 6: The measurement of the phantom with multiple frequencies in length- and transversal direction with consideration of an imprecisely measured outer length, and

FIG. 7: The measurement of the phantom with multiple frequencies longitudinally and transversally with consideration of a precisely measured outer length.

FIG. 8: Shows the side view of the FS—ECG.

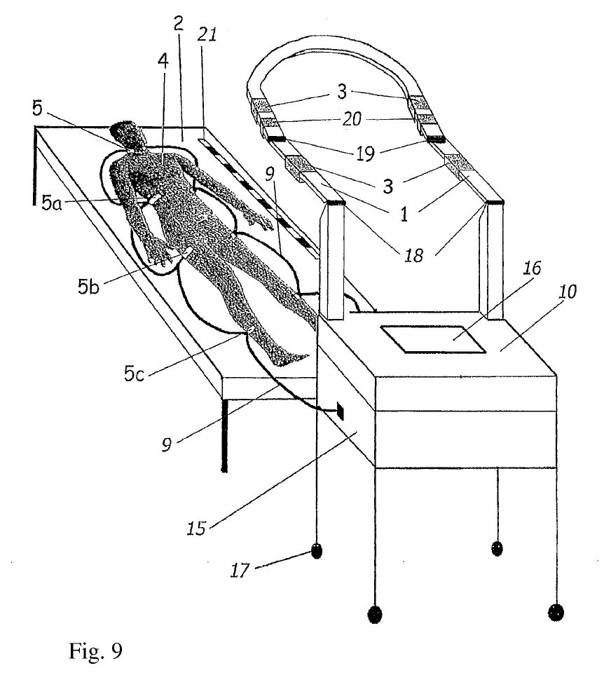

FIG. 9: Shows the FS—ECG three dimensionally.

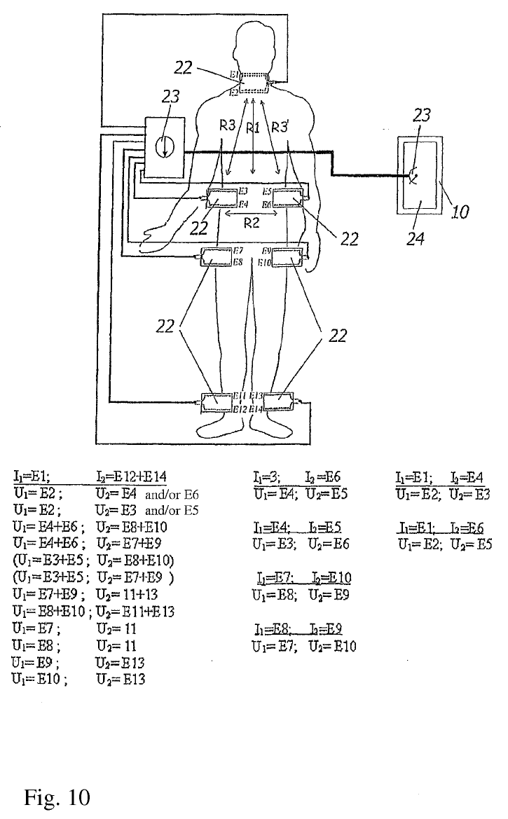

FIG. 10: Shows the preferred positions of electrodes and circuit.

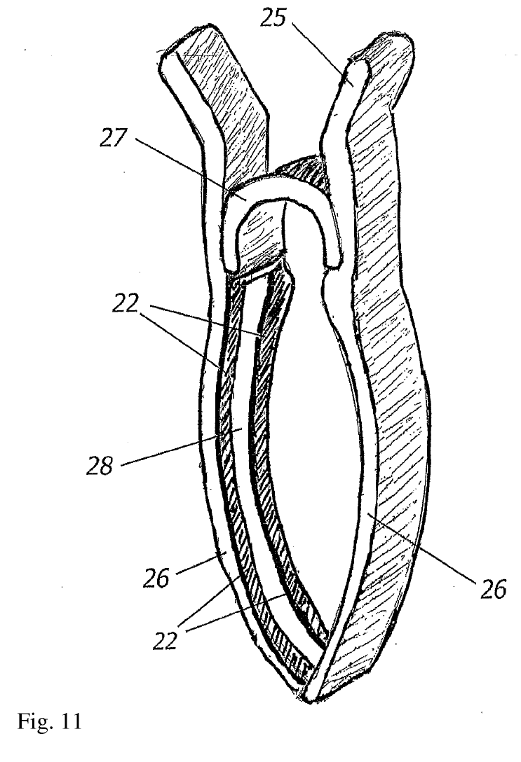

FIG. 11: Shows a clamp electrode with inbuilt double electrode.

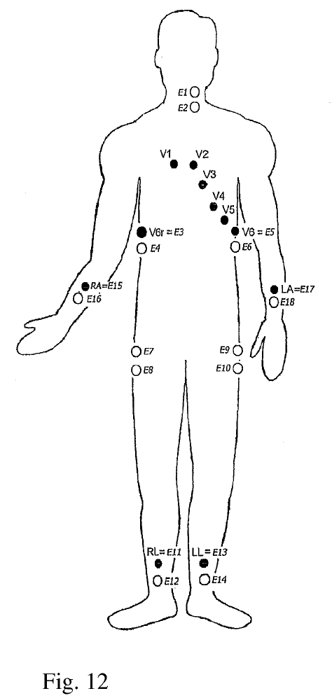

FIG. 12: Shows the body with applied spot electrodes.

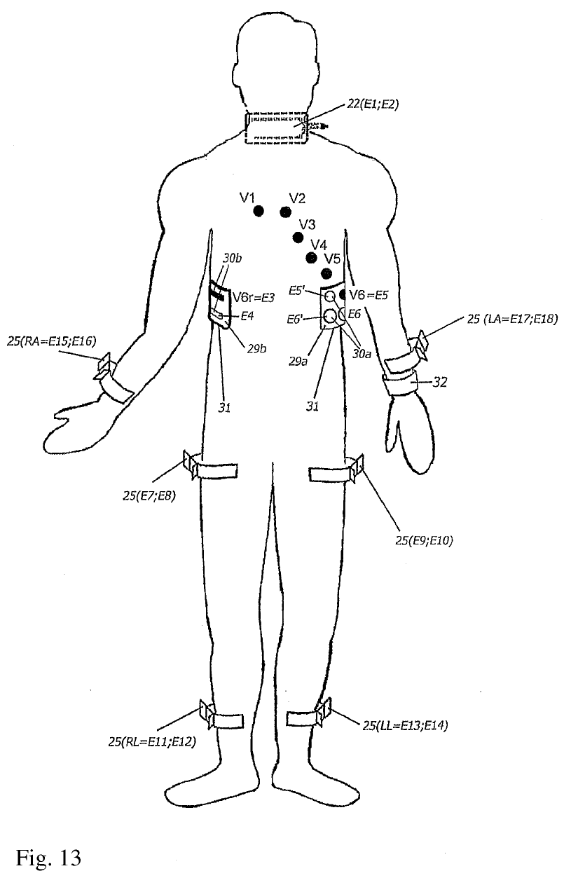

FIG. 13: Shows the body with combined clamp and spot electrodes.

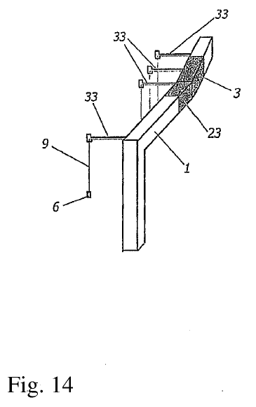

FIG. 14: Shows the instrument arm with side wings.

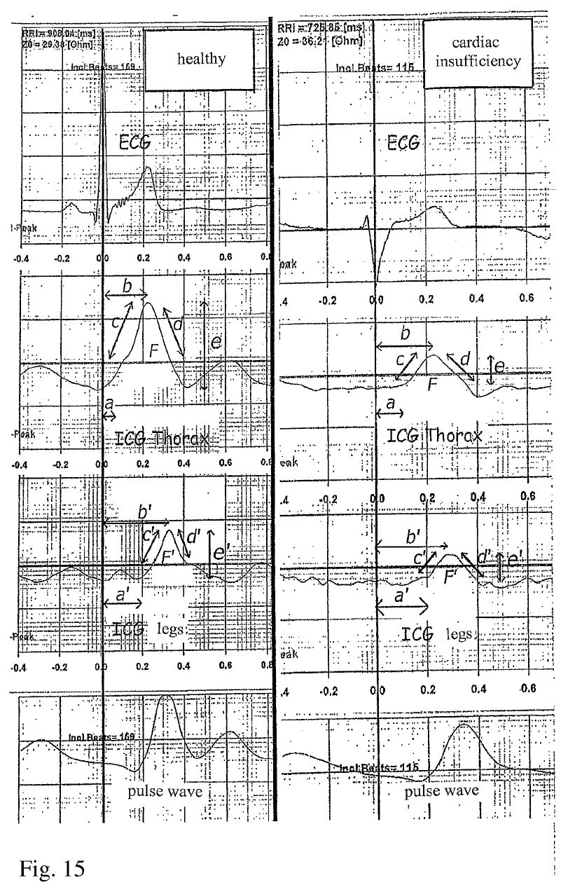

FIG. 15: Shows the impedance signal in two downstream body segments.

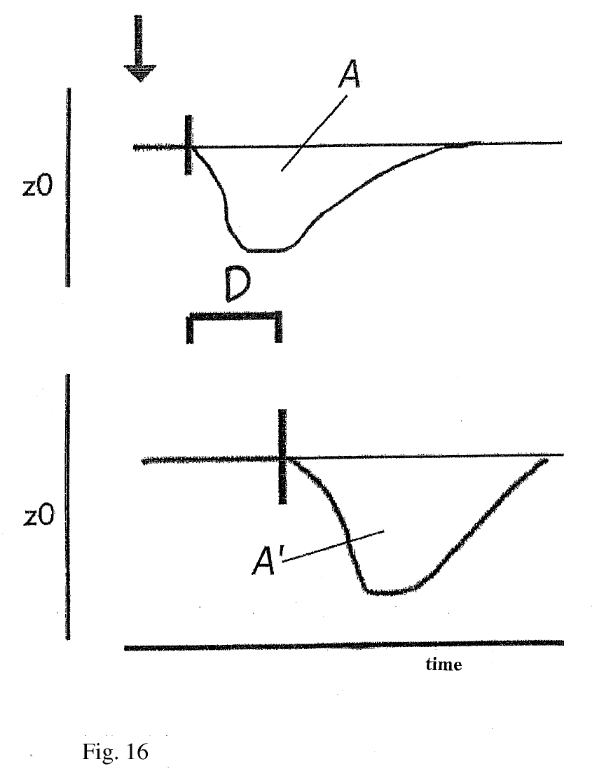

FIG. 16: Shows the impedance change after injection of fluid which has an alternate current resistance different from blood.

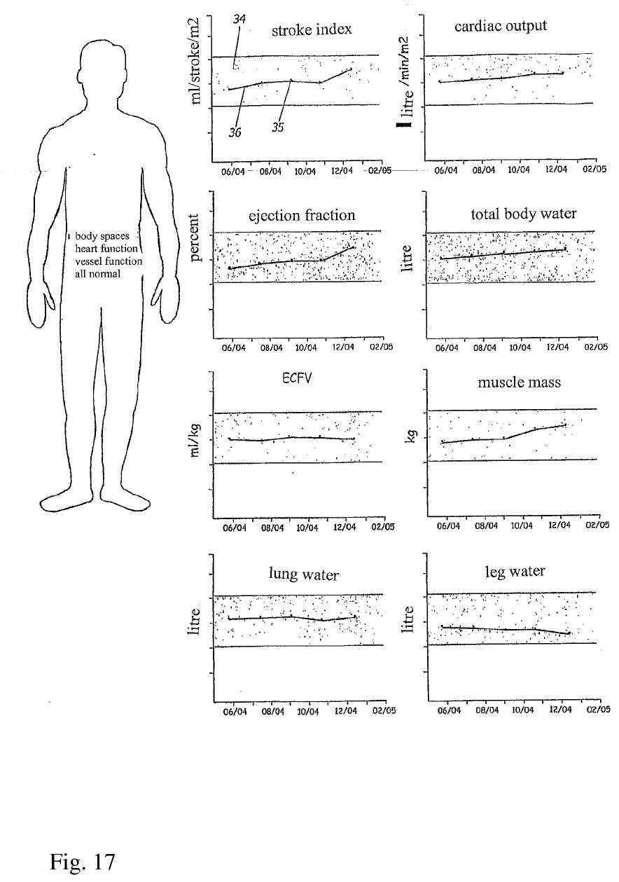

FIG. 17: Shows the report of a healthy subject.

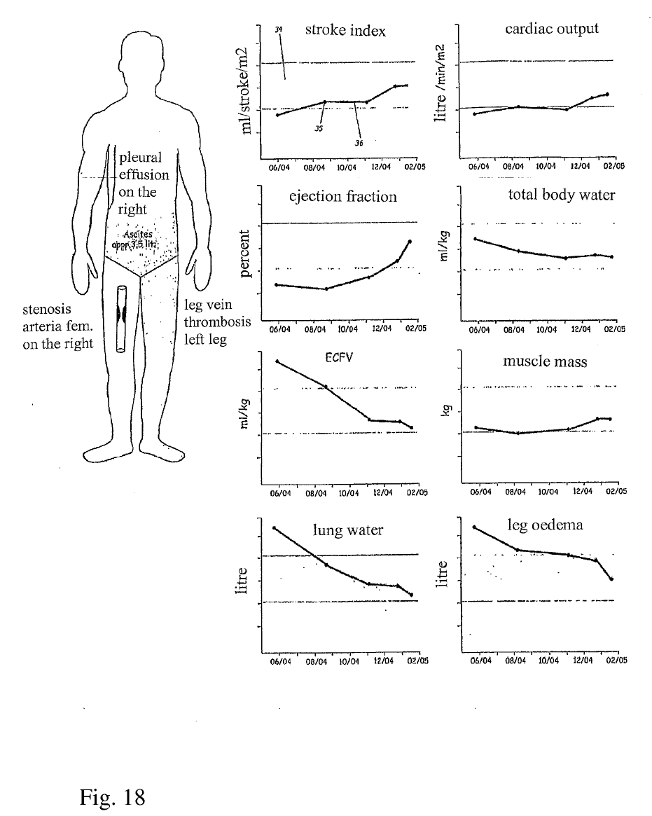

FIG. 18: Shows the report of an ill patient.

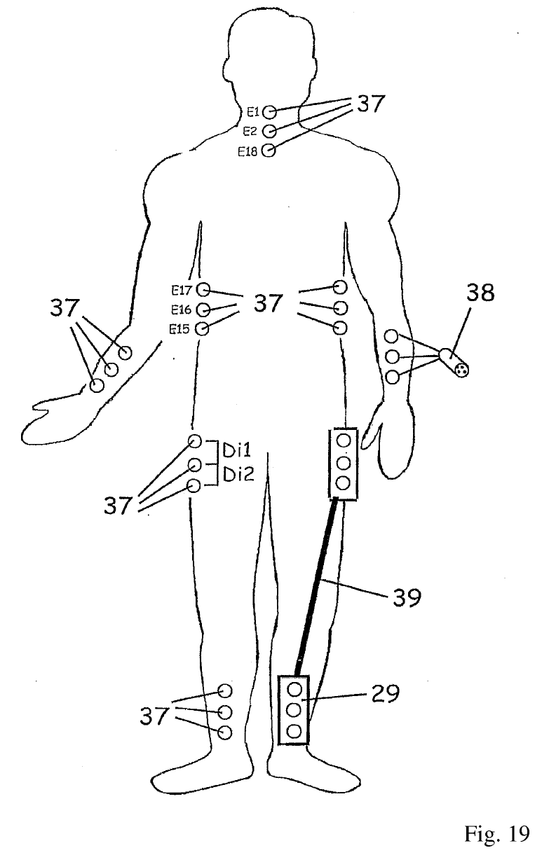

FIG. 19: Shows a further electrode positioning.

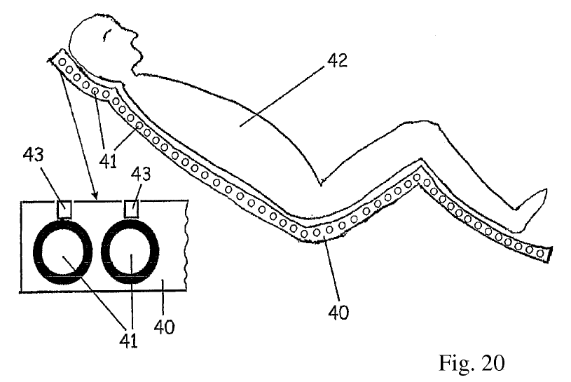

FIG. 20: Shows an examination mat which adjusts to the shape of the body.

The following figures show the results of investigations of the cylinder phantom with three different lengths into which a variable number of non contacting cylinders and discs of different diameters and therefore of different volumes has been inserted, thereby varying the fluid volume within the three cylinder segments (L1, L2, L3) of different length enormously (FIG. 1). Subsequently it was attempted with the help of multiple regression equations to estimate the known volume of fluid remaining within the cylinder, whereby length and diameter of the cylinder phantom were first considered to be unknown (FIG. 2-7). FIG. 2 shows the prediction of the remaining fluid volume (known to the investigator) only on the basis of a length measurement of the impedance at one frequency, FIG. 3 shows the estimation of the remaining fluid volume on the basis of length measurements at one distance and three frequencies, FIG. 4 shows an estimation of the remaining fluid volume on the basis of measurements at multiple frequencies and varying the length measurement by a minute amount as described in patent application PCT/AT 03/00302. FIG. 5 shows the estimation of the fluid volume on the basis of multiple frequencies measurements with one length and one transversal measurement of impedance. FIG. 6 shows the approximation of the remaining fluid volume on the basis of two length and one transversal measurement of impedance at multiple frequencies, in addition the length of the segment was provided with an accuracy of ±2 cm. The ±2 cm were an optimistic assumption of the accuracy achievable in vivo. For that purpose the true dimensions of the cylinder were varied by a random generator within the given ±2 cm, a dimension which can hardly be achieved in practice. Even the bad precision of ±2 cm is in practice hard to achieve due to the complexity of the human body, therefore this is not a realistic but an extremely optimistic assumption. As can be seen from FIG. 2-6 the possibility to predict the remaining “unknown” fluid volume improves considerably, if the cylinder with its extremely inhomogeneous fluid distribution is measured at different frequencies and at two different lengths or if it is measured longitudinally, and transversally.

As can be seen (FIG. 5), when estimating the volume, the transversal measurement of impedance is clearly superior to the measurement of impedance at two slightly different lengths (FIG. 4). The utility of measuring the phantom at multiple frequencies is astonishing, since the cylinder was filled only with one single electrolyte and therefore there was only one compartment present. This certainly is to be seen, among other things, in connection with a therefore better ascertainment of the inhomogeneities. This underlines the importance of the measurement of each compartment at least two frequencies characteristic for the respective compartment, but different as much as possible. Even better would naturally be a complete frequency sweep over the entire frequency band of e.g. from one kHz to 2 MHz or parts of it. As can be seen the deviation of the estimated from the true remaining fluid volume in FIG. 6 is still ±2.400 ml (±2SD). It must be noticed that in spite of the improvement of the prediction through length- and transversal measurements the accuracy of the method is absolutely insufficient. The coefficient of determination of 0.94 deceives the fact that the volume could only be half or double the amount as estimated, as is shown in FIG. 6. This makes the method unfit for clinical practice. For humans the situation becomes additionally even more complex because parallel and serial circuits of resistance and reactance exist beside each other; in contrast, in the simple in vitro model as shown, the cylinder was filled only with one electrolyte but not with living cells with their cell membranes working as a dipole.

FIG. 7 in contrast shows the rigorous gain of information, how much the estimation of the remaining fluid volume improves if the outer dimensions of the cylinder with cross sectional area and length, the “containing volume” is known up to a millimetre and is introduced into the prediction equation. As is generally known, the body surface can be measured with a precision of less than one millimetre deviation with the help of the described volumetric methods. As seen from FIG. 7 the method now suddenly becomes sufficiently precise for the clinical application since the error is now less than 1.058 ml (±2SD) of the measured value, whereby, only in this case, most values notably lie within ±529 ml and only single outliers cause the high 2 SD. The different length- and transversal measurements of impedance contribute still highly significantly to the prediction, even with accurate knowledge of the outer dimensions of the true length of the cylinder as is shown in the following table:

| Model | T-value | significance (p<) |
|---|---|---|
| Constant | 3.4 | 0.001 |
| Length impedance 5 kHz | −4.3 | 0.000 |
| Length impedance 40 kHz | −9.0 | 0.000 |
| Length impedance 400 kHz | +7.5 | 0.000 |
| Length impedance short 400 kHz | −7.3 | 0.000 |
| Transverse impedance 40 kHz | −6.3 | 0.000 |
| Transverse impedance 400 kHz | +6.3 | 0.000 |
| Accurate outer length | +93.3 | 0.000 |

Thereby it is not considered that, of course, by the knowledge of the true outer cross sectional area of the conductor (a human body) an even better definition of the geometry and hence of the fluid components in this body is possible. Since the cross sectional area of human body parts with their contour irregularities, distortions and bulges is not measurable with a measuring tape, the here demonstrated volumetry will bring a considerable additional advance with the electrical measurement of body spaces. The results presented here underline the importance of the contactless measurement of the absolute volume of the body as is explained in this scripture. Thereby in the future it will be possible to quantify not only the absolute volume of the body, but also much more precisely than previously the volume components of the body, namely water and fat. Knowing the specific weight of fat free mass (ca. 1.1) and fat mass (ca. 0.9), the total body weight can then be calculated even without weighing the patient.

The following figures show the different characteristics of the FS—ECG:

FIG. 8 shows an instrument arm -1-, which e.g. at least momentarily is placed in constant position to an examination table -2- (s. FIG. 2) or which also could be fixed on this. In this instrument arm 1 contactless measuring devices -3- for physical dimensions could be placed. An important dimension is e.g. the volume of the examined body -4-. With the help of the FS—ECG the amount of fluids, e.g. intracellular and extra-cellular volume, are derived. These absolute volumes namely e.g. extra-cellular and intracellular water, leg oedema, ascites, lung oedema, etc., not only can be calculated with greater accuracy (see FIG. 7) but also can then be expressed in true litres and in % of body volume, which is e.g. for the ECF, ICF, TBW the best established standard. By knowing the volume of the examined body and of its water contingent (and therefore also the non-water namely fat contingent), the total weight of the examined body can be calculated very precisely from the specific weight of water of fat. This replaces also the weighing of patients, which is nearly impossible with severely ill patients, since they cannot be put on a standing balance or in many cases not even on a sitting balance.

With -3-, one or possibly more contactless measuring devices 3 for physical properties are shown with which e.g. the volume of the examined body -4- and the position of the placed electrodes 5a, 5b, 5c, 5d and of the associated electrode brackets -6a-, -6b-, -6c-, -6-d- can be captured. These could be e.g. distance measuring devices or optionally additional angle measurement devices as they are known in the technical world and as they can be bought cheaply from serial production. These could be e.g. radio, ultrasound or optical measuring devices which measure in a known way the physical properties of the body like e.g. the volume (which is given from the distance of the body parts in relation to the distance from the examination table). Usually either an interferometry, time of flight or triangulation method is used. So the reflected or dispersed signal could be analysed. In the most simple case only a digital picture of the body and/or of its segments could be produced e.g. by CCD or CMOS in more than one plane in order to calculate the volume of the body and of its segments with the help of special algorithms. Simultaneously the distances between the different electrodes can also be derived in this manner. For that purpose each respective electrode of interest (see. e.g. -5a-, -5b-, -5c-, -5d-) should be recognised by the contactless measuring device(s)-3- by their respective distinctive form. By knowing the distance and the angle of the different electrodes to the distance holder also the distance between the electrodes can be determined at once. By using a characteristic profile of the electrodes or their electrodes brackets (-6a-, -6b-, -6c-, -6d-), the correct application of the electrodes can be recognised immediately. It is important to recognise the true distance between the electrodes because by the knowledge of the true distance a possible faulty hydration in the particular body segment can be recognised even better. This is so important since a false position of the electrode - 5a, 5b, 5c, 5d by only ±2 cm would make a difference of one litre of fluid with a segment diameter of approx. 18 cm (e.g. upper thigh) when the body is examined repeatedly. With a diameter of the thorax of about 30 cm this would mean a difference of already 2.8 litres. This would make the method useless. The contactless surface measurement has an accuracy in the range of one millimetre (e.g. sick ranger or LMS 400 laser), which would produce in the given example an error of 0.07 litre instead of 2.8 litres. Particularly advantageous for the detection of the electrode position are electrodes -5a, 5b, 5c, 5d or the electrode brackets -6a, 6b, 6c, 6d, which can be recognised as unmistakable targets for the surface measuring instrument due to specific physical properties as indicated in FIG. 8 (e.g. profile, colour, special reflection, oscillation, temperature, etc.): Therewith a wrong placement or permutation of the electrodes can be detected immediately and can be signalled to the user if the individual electrodes have a special identifier. So the patient lying on the examination table -2- can be gauged accurately in three dimensions by the contactless measuring device(s) -3-. With the help of the three dimensional picture of the patient the volumes of the different segments can be easily obtained and spurious hydration and the fat portion of the different compartments can be derived. Advantageously the electrodes are not placed in the extreme periphery (that is hand and food) but more centrally on the distal lower leg and on the distal lower arm, in order not to produce unnecessarily a series resistor within the tapering extremities. In order to still obtain later the total volume of the compartments, it proves to be useful to extrapolate the part of the body which lies outside of the electrodes by the contactless volume measurement. In order to scan the total body concerning the distance and other physical parameters, e.g. the contactless measuring device -3- could be swung with a swing drive -7- whereby the angle measuring device and the distance measuring device can still produce a correct picture of the body since the examination table -2- (with a constant preferably flat form), on which the examined body -4- is placed, can be taken as a reference. So the possibly arising distortion of the examined body can be corrected easily mathematically. When swinging the contactless measuring devices with the help of the swing drive -7- it must be considered that the measuring fields -8- of different contactless measuring devices -3 overlap, so that a complete three dimensional picture of the body is possible. The electrodes -5a-, to -5d- or the respective electrode brackets -6a- to -6d- are connected though cables -9- to the FS—ECG -10-. In the figure only the cables of one side of the body but not from the other side are shown. The examination table -2- is important as a reference area, because usually the body must be depicted from all sides in order to obtain the true volume. This however is not possible with severely ill patients, therefore the examination table -2- must be taken as hindmost reference area. This is especially possible if the area is of relatively hard consistence, because then it will not change its form appreciably. Therefore the hindmost area of the body which is not depicted can be taken as relatively flat. But even if it is not as rigid, it will be captured three dimensionally in the border zone between body and examination table -2- how the table deforms under the weight of the body, so that the volume of the body can still be calculated accurately. Especially the elastic modulus of the table could be known, which is used for the calculation of the volume of the body -4-. On the other hand the contactless measuring device -3- could also be moved with a thrust drive -11- along the instrument arm -1-. The thrust drive-11- could be an x and/or an x/y drive, in order to move the contactless measuring device -3- with constant measuring angle -12- over the body -4- in order to obtain an accurate three-dimensional picture of the examined body and its segments. The thrust drive -11- is only shown for one of both depictured contactless measuring devices -3-, it would of course be necessary for all of them unless an unmoved contactless measuring device -3- without swinging or thrusting produces a complete 3D picture of the body. When applying this method it has to be considered that by using an ultrasound usually a cone-shaped dispersion is produced, whereas with optical, laser- or radio signals the bundle can be well focused. Therefore possibly optical methods, possibly in the invisible region, possibly in the infrared region are preferable.

If the contactless measuring devices -3- are equipped with other sensors, other physical differences like variations in body temperature between different body parts can also be recognized immediately (thermography) and can be used for diagnosis. Only local warming through inflammation or local cooling caused by local under-perfusion are named as examples. Additionally the method of active thermography is named for example, in which the conductance of temperature by the surrounding tissue is examined. This allows the slightly easier recognition of fluid accumulation in the underlying tissue like e.g. by hydrops or effusion.

The examination table -2- has advantageously a table upper part -13- projecting above the horizontal plane, as shown in the figure, since often patients with heart failure are examined, who often cannot be positioned horizontally. The angle of inclination should be advantageously between 20 and 45°, e.g. also 30°. An automatic or manual mediation of the inclination angle -14- of the table upper part -10- should be available. Also an inclination for the lower body half projecting below the horizontal plane could be of advantage. At a fixed inclination angle the measurement of the described physical properties of the patient is facilitated. In order to compare measured values of the same patient at different points in time the patient should be examined at the same inclination angle, otherwise fluid shifts would alter the measured values.

Also the examination table could mediate physical values which cannot be captured with the contactless methods described above. So e.g. one or several pressure sensitive sensors could be built into the table or into a mat respectively, below the patient, which capture the weight of the patient or of his body segments (not shown).

As shown in FIG. 9 the instrument arm -1- could be mounted at a measurement trolley -15-. This would be more advantageous since by the placement of the FS—ECG -10- with the display -16- on the measurement trolley -15- this could be moved easily with the existing rolls -17- from patient to patient. For the transport it is important that the instrument arm -1- with the help of the swivel arm -18- could be brought into a vertical position preferably lengthwise to the measurement trolley -15-. The instrument arm -1- should be equipped with additional joints -19- (e.g. telescope mechanism or bend-mechanism etc) which shorten it in a way that it does not obstruct during the transport of the measurement trolley but that it is in line with the measurement trolley -15-. The cables -9- from the FS—ECG -10- to the body -4- are designed e.g. as bundle cables, in order to disturb the 3D capture of the body -4- as little as possible.

In practice it will suffice not to shift or rotate the contactless measurement device(s) -3-, but to capture the body three dimensionally with multiple measurement devices simultaneously as shown in FIG. 9. In this figure the instrument arm -1- is designed twofold in order to better obtain a three dimensional image of the body. The contactless 2D or 3D measurement devices -3- are e.g. each realized twice in the U-shaped instrument arm 1. Therewith a complete 3 D image of the body can be obtained very rapidly, whereby the positioning at the side of the body -4- ensures that the critical border zones between body -4- and examination table -2- are depicted particularly accurately and e.g. a possible deformity of the examination table -2- is recognized. The equipment with e.g. simple and cheap CCDs or CMOS as contactless measurement instruments -3- or the like together with a grid projector -20- or e.g. infrared grid or point or line projector could enable the unnoticeable calculation of the body volume and its segments within seconds. This would be particularly important because motion artefacts of the body -4- are excluded completely. A simultaneously depicted ruler -21- enables the consideration of a different distance between instrument arm -1- and examination table -2- at any time. On the other hand the U-shaped instrument arm -1- could serve to use e.g. a single contactless measurement device -3- with the thrust drive -11- (see FIG. 8) or by pivoting, for depicting the body -4- from different positions, which however has the disadvantage of a time delay of the measurements.

Should the 3D measurement of the body serve to simultaneously obtain with the help of impedance the extracellular volume and intracellular volume of the body, the measurement of the nearly cylindrical body segments longitudinally and transversally needs a special electrode positioning which is described in the figures below in more detail.

FIG. 10 shows a possible placement of the electrodes for introducing the current and for the voltage measurements which is suited for exact capture of body volumes, of heart action and of perfusion of different body segments. With -22- the used double electrodes are indicated, which are here e.g. executed as bands. They could be executed just as well in arbitrary shape like e.g. as spot electrodes. With -23 a current inducing change over switch and change over switch for placing the measurement sites (short: switch) is shown, which ensures that the at any time correct segment is either measured longitudinally, transversally or diagonally (by switching off one of the electrodes at the identical body height) and with which optionally also the current induction could be switched. With -10 the FS—ECG is shown, which also contains the constant current source and the impedance meter -24. This could be a multiple frequency impedance meter including an alternating current source. This could e.g. produce and measure discrete frequencies of e.g. 5, 40, 200 and 400 kHz or also a complete frequency sweep over each desired measurement range. The favoured electrode positions are signified with E1 to E14. E1 and E2 are placed on the neck, E3 to E6 on both sides at the thorax respectively, E7 to E 10 on both sides at the beginning of the leg respectively and E11 to E 14 respectively at both distal calves. It would be advantageous to execute all electrodes in a similar way in order to simplify the handling. So all electrodes namely ECG and impedance electrodes could be executed as adhesive electrodes. For short term measurements it could prove to be advantageous to use a weakly adhesive glue, which can be easily and painlessly removed from the body, similar as it is known e.g. from post it labels. On the other hand all electrodes could be executed as suction electrodes, as is known from the ECG and usual. For long term measurements or indefinite measurements well adhesive electrodes will be advantageous.

In the lower part of FIG. 10 the most advantageous combinations of current and voltage switching are shown, which can of course be supplemented if necessary. For the measurement in the horizontal plane, one electrode of the double electrode should advantageously be the current electrode, the other one the voltage electrode. Since a true four point measurement with current electrodes being positioned outside and voltage electrodes being positioned inside horizontally on the trunk, abdomen and on the legs is hardly possible, it is proposed to switch each of the double electrode pairs, being positioned at the same level of the body, diagonally and alternatively as current and voltage electrodes, as is shown in the right-hand lower part of FIG. 10.

The mean value of both measurements in the respective diagonal direction is a good measure for the horizontal impedance and therefore for the transversal fluid content of the respective body segment. Furthermore it is shown that it could be advantageous to introduce the current via the electrodes between the electrode position E1 on the one hand and E12 and E14 on the other hand and also to measure the voltage a) between the electrode positions E2 and E3, b) between E2 and E4, otherwise between c) E2 and E5 and furthermore between d) E2 and E6. Thereby the thorax will be measured diagonally at respectively two different thorax lengths in the directions R3 and R3′ in order to recognize immediately a one sided or two sided pleural effusion. In addition it is also considered to verify electrically the correct position of the electrodes and the correct wiring of the electrodes before beginning the measurement. This is easily possible since, with correct wiring of the double electrodes, the outer electrode farther remote from the centre of the body must show a higher resistance to the alternate current than the inner electrodes lying closer together. So even after false placement of the electrodes the falsely wired electrode can be wired correctly later. The described design is much more economical than the use of multiple impedance meters, which, of course, would be possible as an alternative.

The multiple frequency impedance meter including the source of the alternate current is shown by -24- as part of the FS—ECG. This can produce and measure e.g. discrete frequencies of e.g. 5, 40, 200 and 400 kHz or a complete frequency sweep over any desired measuring range.

FIG. 11 shows a further execution of the electrodes whereby an elastic clamp -25- is placed on the body parts, which has on its opposite frames -26- at least one or two double electrodes -22- which through the tension device -27- of the elastic clamp -25 find good contact to the body. The frames -26 should be contacted in a way that an insulation between the double electrodes -22 is ensured. Thereby e.g. the frame could be equipped with only one deepening -28- between the double electrodes which has no contact to the body -4-.

FIG. 12 shows the human trunk where the same electrodes which are executed as band electrodes in FIG. 10 are now executed as spot electrodes. The numbering corresponds to that shown in FIG. 8. In addition, however, the conventional ECG placements are shown in black whereby RA corresponds to the electrode on the right arm, LA to the electrode placement on the left arm, RL to the electrode placement on the right leg, LL to the electrode placement on the left leg. Advantageously the leg electrodes RL, LL and E11 to E14 respectively are placed on the calf muscle and not further distally because further distally the ill conducting tissue would cause a series resistance which would unnecessarily and uncontrollably increase the impedance. Subsequently, the contactless volume measurement can calculate the volume of the body part located outside of the electrodes which then can be calculated and added to the body spaces.

V1 to V6 show the conventional chest lead electrodes. V6 can also be used as ICG electrode position, E5 (or E6), RL as ICG electrode position E11 (or E7, E8, E12), LL as ICG electrode position E13 (or E9, E 10 or E14). Also the ICG electrode position E3 could be used additionally as ECG electrode position V6r. As is known, these electrodes could be executed e.g. as suction electrodes in connection with a vacuum source (not shown) as it is often usual nowadays. Thereby e.g. also electrodes which have to be placed in constant position to each other like the electrodes V6 and E6 shown here could be placed on a common carrier -29 so that the correct distance of the adhesive or suction electrodes is guaranteed in any case. Also the ECG electrode positions RA und LA could be used to measure also the impedance of the arm segments whereby RA e.g. could be used as additional ICG electrode position E15 (or E16) und LA as additional ICG electrode position E 18 (or E 19). The application of the current could be performed through the electrode position E 16 and E1 on the one hand and E18 and E1 on the other hand, the voltage measurement respectively between E2 and E15 on the one hand and between E2 und E17 on the other hand. Also for the other electrode positions which are to be placed in constant distance to each other a common carrier could be provided (not shown).

FIG. 13 shows probably the most profitable execution of the electrode position in practice, because it is very similar to the conventional ECG and therefore no reeducation of the staff is required. Thereby reusable elastic clamps -25- could be used in a known manner for economical reasons (whereby those contain now double electrodes as shown in FIG. 11) and on the thorax suction electrodes could also be used for economical reasons, which are familiar to the staff already from the conventional ECG. It is also shown that the double electrodes (FIG. 10; -22-) at the thorax can also be executed on a common carrier -29 as double suction electrodes -30a-, -30b-. 30a shows that more than one e.g. four spot electrodes are placed on a common carrier -29- (e.g. suction electrodes) in order to ensure a reproducible distance between the electrodes (E5, E6). If the electrodes E5 and E5′ and respectively the electrodes E6 and E6′ are connected electrically to each other the effect of a nearly band shaped electrode emerges. On the other hand the suction electrode could also contain two nearly band shaped electrodes as indicated by -29b. The sealing to the body is indicated by the symbol -30- whereby naturally each single electrode could be designed as a separate suction device in a known manner. For the follow up of patients in many instances peripheral ECG leads will suffice, so that the thorax electrodes V1 to V5 could be omitted, only the double electrode -22- on the neck could be used as a disposable adhesive electrode in order to create disposables. The neck electrode should then, for example, be produced in such a way that it is certainly suitable only for one-time or limited use. For that purpose e.g. also a barcode could be present, which must be imported or electronic protection could be integrated in the electrode, which enables the FS—ECG to recognize immediately whether this electrode has been used already respectively which prevents the use of non-licensed electrodes in advance. For the purpose, e.g. RFID (radio frequency identification) would be an option. Also every other electrode at the body could be (with less purpose) executed in that way. If the production of an electronic code for the electrodes is too elaborate, one could instead use a barcode reader or any other code reader for the FS—ECG.

Over and above, a pressure cuff -32- is shown at the wrist, which can be used for conventional oscillometric blood pressure measurement or as shown in the patent (European patent application 05000042.1, publication number 1522258 or AT 391.262B) could contain a fluid filled bladder over an artery. This could be pressurized with a variable pressure, so that an automatic blood pressure measurement and pulse wave analysis could be performed as shown in the above-mentioned patent application. From this e.g. central aortic blood pressure could be calculated, which is so important but never measured routinely. This important parameter is in practice not measured because this would require additional time and staff. The micro processor controlled fluid bladder (as shown in European Patent Application 0500042. 1, publication number 1522258) can perform this alone in the background, so that this important parameter can be obtained simultaneously without any additional personnel expenditure.

As shown in FIG. 14 the instrument arm -1- could also be used for leading the cables -9- to the patient whereby these could be led to the body from different positions of the instrument arm, in order to prevent a disarrangement of the cables -9- which otherwise would be hardly avoidable with a great number of cables (e.g. leg electrodes from one end of the instrument arm, head and thorax electrodes from the other end of the instrument arm). The mechanical or electronic switches -23- are advantageously also located in the instrument arm indicated by -1-, which switches are actuated by a central micro processor, advantageously located in the central FS—ECG -10-, and connect the FS—ECG -10- to the respective electrode positions -E1 to E14, optionally to E18. Minimally only two current and two voltage cables, the actuating cables and the shielding have to lead from the FS—ECG -10 to the instrument arm -1-, which carry the possibly pre-amplified signal. This has the advantage of better overview and also that the contactless measuring devices -3 located in the instrument arm -1 are not disturbed to the cables -9. For that purpose e.g. the instrument arm could be equipped also with side wings-33- for the cables -9, which remove the cables from the measuring field covered by the contactless measuring instruments -3-. The cables -9 originating from the instrument arm -1- would have also the advantage that the cables -9- leading to the patient, which are advantageously shielded, could be of equal lengths. The shielding could also be executed as an active shielding.

Should a single bundled cable -9- be used, as is shown in FIG. 8 and FIG. 9, from which all electrode connections originate, the danger of disarrangement of the cable(s) is markedly reduced and the side wings are no longer needed. In order to make the instrument also transportable it is proposed that all parts protruding from the FS—ECG -10- and from the measurement trolley -15- like instrument arm -1- or side wings -33- are either executed as telescopic parts or can be swung ideally in short segments to the measurement trolley -15-. For the distance measurement between the electrodes alone, the at least one contactless measurement device -3- could be mounted also to the electrodes -5a- to -5d- placed on the body or to their electrode brackets -6a- to -6d-, with which the contact to the cable -9- is carried out. This would have to be executed as a distance measuring device (e.g. using one of the above methods). (No figure).

As is shown from FIG. 8 to 14 it needs a great number of different measurements at the electrodes which are already routinely present, which can only be accomplished by means of multiple switching and/or by multiple impedance meters. The final specification will depend on technical and financial considerations.

It is to be minded in any case that in spite of the multiple leads that are needed, the usual protection required for the license is given for the patient and the examiner even in case of application of electrical impulses e.g. in case of cardioversion or defibrillation and also in case of a malfunction of the FS—ECG -10-, in order not to endanger the patient. The operating expense in technical and time terms is trivial despite the multiple switching operations, since for the greater part of the leads only basal impedance z0, but not dz or dz/dt with the heart beat need to be recorded.

Since the switching procedures occur automatically, advantageously micro processor controlled, neither the physician nor the patient notices the complexity of the method. The result can be obtained within minutes. Also by comparing the measured impedances it can be easily recognized if electrodes are displaced and a respective warning can be given. Also a variable configuration of the multiple contact plaques could guarantee that only the correct cable -9 is connected to the correct electrode. The use of conventional colors of plugs and associated elastic clamps (RA=red, LA=yellow, LL=green, RL=black, and new: left upper thigh=light green, right upper thigh=grey) makes the operation smooth also for untrained staff.

FIG. 15 shows the impedance signal, which is registered simultaneously at the thorax (upper part of the figure) and on the legs (lower part of the figures). On the left side of FIG. 15 the signals obtained from a healthy control are recorded. As can be seen the change of impedance (z) over time with the heartbeat, namely dz/dt at the legs, is delayed with an interval -a′- compared to the thorax -a-, which has to be expected physiologically. The time delay of the steepest slope of dz/dt between the thorax and the leg segment allows important conclusions about heart and vessel function, since a congestion in the peripheral circulation has an impact on the interval (a, a′, b, b′), on the shape of the curve with a change of slopes -c- and -c′- and on down sloping -d- and -d′- of the impedance signal, furthermore of the amplitude -e- and -e′- and of the area -F- and -F′-. This is clearly to be seen from the differences between the healthy subject (left) and the patient with heart disease (right side of the figure) which can be recognized at the first glance. Furthermore a complex signal analysis with gauging of all areas and of curve progression will be helpful. Therefore the dz or dz/dt signal of the thorax and of the legs will enable much better to draw a conclusion about the actual heart performance than the measurement of the thorax segment alone. Especially the part of the dz in the thorax, which is altered by a reflected pulse wave can be detected and can be corrected for this reflected part. The similarity of the respective dz in thorax and legs leads to the conclusion that the interpretation of the thorax dz has so far been much too complicated. This can be established since, without the existence of a left and right heart or of atria or of the large and the small circulation, the curve in the leg is similar to the thorax curve. Knowing exactly the total volume in a segment it could now suffice to measure the total dz and to subtract only that part of dz of the thorax which is derived from the legs and other parts of the periphery in order to accurately calculate the stroke volume. Furthermore it is now for the first time possible without the use of mechanical transducers to measure pulse wave velocity in the aorta accurately on a purely electrical basis. For that purpose the contactless measurement device -3 for deriving the body surface is also helpful because the distance between thorax and the legs can be measured down to a millimeter. Only the pulse wave velocity in the central aorta (but not in the arm or leg segment) gives important information about vessel properties and correlates with the prognosis of disease of the heart and the circulation.

From the different amplitudes of the dz or dz/dt and from a form analysis of the impedance curve of the left and right leg important conclusions about arterial perfusion, differences in perfusion between both legs and about impaired venous return can be drawn. Optionally, this could be further improved by the use of pressure tourniquet on both legs, whereby the impedance measurement is performed as venous occlusion plethysmography. An increase in the pressure of the tourniquet above the venous pressure or also near to the arterial pressure makes the method with the help of the accurate volume measurement more versatile as a plethysmographic or oscillometric method.

In FIG. 16 a change of impedance in the upper thoracic segment and in the lower thoracic segment after intravenous injection of a bolus of fluid, which changes the impedance of blood, into an arm vein is shown. For the measurement of this impedance ramp e.g. the current could be applied at the electrode position on the neck -E1- and at the electrode positions of the legs -E12 and E14 (s. FIGS. 10 and 12), the chest electrodes -V1 to V6 could optionally be used for the measurement of the impedance ramp, e.g. also an impedance ramp, which is time delayed between V1 and V2 on the one hand and between V5 and V6 on the other hand, which is marked as -D and which will be a measure of heart performance. Also the area under the respective curve -A and A′ and also the curve shape caused by the injection of the medium with conductivity different from conductivity of blood will give important information about heart function according to the Fick principle. Also other segments of the body could be used for the measurement of the impedance ramp and of the curve form. Use of this principle is of course possible in any additional eligible regions, e.g. also in the leg segment and also in the region of the arterial blood stream.

It is obvious that because of the marked complexity of the human compartments, the suggested multiple measurements are necessary, on the one hand, for defining said compartments. On the other hand excellent clinical evaluation and calibration data in a great number of patients will be necessary in order to be able to introduce the method into clinical medicine. The improvement in the estimation of complex compartments is shown clearly in FIG. 2 to FIG. 7. Only the best result with use of multiple measuring points at multiple frequencies and only the knowledge of the true dimensions of the examined body segment as shown in FIG. 7 will be accurate enough for clinical use.

FIG. 17 and FIG. 18 show an example of a possible report of a function & spaces (FS—ECG) from which it is seen that all important haemodynamic and fluid data can be displayed in numerical and graphical form including text modules. Simultaneously the data of the patient are saved in a data bank in order to immediately compare the results with previous measurements, which advantageously should be presented in graphical form. Thereby the normal values can be e.g. displayed in form of normal range fields -34- in which the data of the patient can be presented as reading points -35-, respectively for the observation of time course through trend lines -36-, which connect the reading points -35-. On the x-axis the time and on the y-axis the value of the observed variable should be shown. So at one glance the success of the therapy induced by the physician can be evaluated and the therapy could be further improved in future.

FIG. 19 shows a further preferred electrode design e.g. positioning on the human body. Thereby all borders of the investigated segment are engaged e.g. with triple electrode elements -37. This electrode design makes it possible to apply the current only into the investigated segment and allows simultaneously to slightly vary the length of the segment measured between the voltage electrodes on both ends. Using this design enables to get a better grip on the so-called border zone phenomenon. Ideally a segment would be examined such that the imaginary outer cross sectional areas of the examined segment would be covered evenly by electrodes for measuring the voltage, which are distributed across the entire area. This is of course not possible non invasively for a human body, therefore one has to be content with surface electrodes on the skin. However, at the edge of the segment it is possible to tap only a part of the voltage active in the segment, which, of course, is particularly small if below the voltage electrodes poorly contacting tissues like bone, tendons or fat are located. However, if the current is brought into the body near the place where the voltage is recovered a greater and more representative part of the voltage will be recovered. So with nine to eleven triple electrodes a very representative image of the fluid distribution in the body can be obtained. Naturally also the leg could be divided by further electrode elements into a thigh and a calf segment, respectively only the calf or only the thigh could be measured. The great numbers of switch over (up to ten or more different current applications and up to 30 or more different voltage measurements) occur fully automatic and unnoticed by the user. Also a design of the electrodes shown in FIG. 19 as band electrodes is of advantage. Also a measurement of whole body impedance with the advantage of the contactless volumetry is, of course, possible. The distances between the electrodes, which define a segment, are detected also fully automatic and unnoticed. It is apparently of advantage to provide each of the shown electrode positions consisting of a triple electrode element -37- with only one distinctive plug in or squeeze connection (e.g. multi plug -38-). The distance of the electrodes at the boundary of each segment is can given either a) through the mounting of the electrodes on a common carrier -29-. It is referred e.g. also to the design as a suction electrode -30- in FIG. 13 or to the elastic clamp -25- in FIG. 11, whereby only triple electrodes instead of double electrodes are now available. On the other hand the process employed for the depiction of body surface e.g. through 3D-photogramometry can also be used to automatically detect the distances Di1 and Di2 of the electrodes within the triple electrode element -37-. In that case a common carrier -29- for the electrodes is not necessary. Also a distance provider -39 for the in each case two double electrodes -22- or triple electrode elements -37, which border the investigated segment, would be conceivable, then also the surface distance between the voltage electrodes from the upper to the lower end of the segment would be known and this without imaging procedures. As an example the examination of all shown or also additional segments (e.g. thigh and calf separately) is shown on the basis of the electrode positions E1, E2, E15, E16, E17 and E18, the current here is only introduced e.g. at the positions E1 and E15, the voltage is measured alternatively between E16 and E2, or between E17 and E2, or between E16 and E18, or between E17 and E18. Therewith e.g. electrically operative lengths as named in the paper of Skrabal et al could be calculated and the electrically participating volume of the segment could be derived with much more precision⁷. All other segments in FIG. 19 or additional segments (e.g. thigh and calf separately) could be gauged in a similar way to the shown segments. For the calculation of the volumes of the segments shown in FIG. 19 all the advantages of a calculation based purely on physical principles without any assumptions can be used: So the segment length will be detected automatically and without errors, the cross sectional area of the segment can be measured accurately to the millimetre and sliver for sliver. This is of drastic importance for the resistance of the segment because e.g. a diminution of the segment e.g. at the knee joint will cause a disproportionate increase of resistance, which now can be calculated accurately according to exact physical formulae; with the help of the frequency sweep e.g. using the Cole-Cole plot the resistance can be calculated accurately at the zero frequency and at the indefinite frequency^{xiv}. By using the Hanai Mixture Theory^{xv}, e.g. also the second generation Hanai Mixture Theory^{xvi} the influence of formed elements (body cells) on the resistance of the segment can be calculated exactly in a physical manner. Also the resistance of the segment according to the formula

Rs=ρ*(L₁/A₁+L₂/A₂+L₃/A₃ to Lₙ/Aₙ)

whereby Rs=resistance of the segment
ρ=specific resistance of the conductor
L₁ to Lₙ=lengths of the respectively investigated slivers of the segment
A₁ to Aₙ=area of the respectively investigated slivers of the segment
can be interpreted mathematically correct for the first time and the erroneous assumption of cylindrical conductors (and this with an even approximated circular cross sectional area) can be refuted. By using the now known anatomy of the segment (see below) each sliver can be attributed with a specific resistance (ρ) which corresponds to its anatomy. This moves the method from an empirical estimation to a comprehensible clearly physically based method. With the help of the exact volumetric measurement of the body also the whole body impedance measurement can be improved considerably because the crude correction factor^{xvii} for the different diameters of arm, trunk and leg segment, which so far has been used for this method, can be replaced by an accurately measured physical magnitude because the diameters of the slivers of arm, trunk and legs are now known exactly for the first time. For the first time it is now also possible to introduce the knowledge of the anatomy into the theoretical model, as a small example it should be mentioned that the knee can be identified as such through the volumetry. Now it is known that at the height of the knee joint nearly exclusively only bones, cartilage and connective tissue is present, all tissues with poor conductance. On the other hand, proximally and distally from the knee joint large muscle masses with very good conductance, especially at high frequencies, are present. In the future also the knowledge of the anatomy will be used for the calculation of intra and extra cellular water on the basis of the practical anatomical model. Considering the number of used electrodes it will be particularly advantageous that only electrodes are used which come from the producer of the FS—ECG, since as consumables these guarantee a steady business volume. Therefore it could be advantageous to provide a code either for the electrodes themselves or for the packaging. This code could be fed manually into the FS—ECG device. Only if the code number of the electrodes agrees with the code numbers stored in the FS—ECG, the instrument would be ready for use. ^{xiv}Cole K S, Cole R H Dispersion and absorption in dielectrics I. Alternating current characteristics J Chem Phys 9: 341-51, 1941 ^{xv}Hanai T. Electrical properties of emulsions. In: Emulsion Science, Ed Sherman P H. London Academic, p 354-477, 1968^{xvi}Mathie J R. Second generation mixture theory equation for estimating intracellular water using bio impedance spectroscopy. J Appl Physiol. 99: 780-81, 2005^{xvii}De Lorenzo A, Andreoli A, Matthie J, Withers P. Predicting body cell mass with bio impedance by using theoretical models: a technological review. J Appl Physiol 82: 1542-58, 1997

Furthermore a special design of the surface on which the patient rests could be of advantage: this design could either be part of the examination table -2- or it could also be particularly advantageous if the patient should not or cannot be moved from his bed to the examination table. FIG. 20 shows a deformable mat -40-, whose deformity is defined and ascertainable. This mat is moved under the patient, either rolled or shifted, so that the patient can remain in the bed. It is especially advantageous if the mat deforms not or only to a minor degree in the transversal direction of the human body but is very good deformable in the length direction of the body. Herewith the mat can be arranged according to the bending and straightening of the different joints (knee joint, hip and spine). The extending part of the mat -40-, which protrudes on the side of the examined living object -42-, will be recognised by the contactless imaging procedure and is used as a reference area (see FIG. 9). If this reference area is representative also for the part of the reference area which is below the human body, the hindmost borders of the human body, which are not depicted, can be derived accurately. FIG. 20 shows a possible design of the mat in the transversal section: thereby, preferably rounded, three angled or multiple angled rods -41-, which are very stable (e.g. tubes -41-) are introduced into a deformable mat -40-, e.g. made from foamed synthetics, thereby the mat -40-can follow the bends of the body. Despite this, through the part of the mat protruding on the side of the examined living object, the hindmost borders of the human body are known accurately, because this mat -40 is identified with the help of the contactless measuring devices exactly as a reference area (see FIG. 9). With the help of e.g. four stereo cameras (consisting e.g. of 8 CCDs) (see FIG. 9) the deformable mat -40- serving as a reference area and the examined human being can be imaged accurately in three dimensions. Below the mat there is a conventional stretcher or bed, in which the patient lies, the form of the mat is determined by the weight of the examined subject, if below the mat a soft deformable material is present (e.g. foamed materials, soft pillows, etc, not shown), another design of the mat could be that the deformity of the deformable mat -40- along the transversal axes could be exactly defined. If the rods e.g. should have a defined deflection radius, one could calculate the deflection of the part below the examined human body from the tubes or rods -40- protruding on the side of the examined body. This would have the advantage that the deformable mat -40 fits even better to the contours of the body and therefore the borders of the examined body in the area which cannot be depicted by the contactless measuring method are defined clearly from the curvature of the deformable mat. The placement of the contactless measuring devices excludes of course an imaging of the examined body from all sides. So in the visible part the contactless measuring devices -2 and in the invisible part the defined bending of the deformable mat -40- define the volume of the examined body and of its segments. Besides the deformable rods -41- also other means for the defined distortion are to be thought of. After use the mat can be rolled together and can also be cleaned easily, herewith an examination of the subject is possible wherever he lies at the moment. Also the equipment of the surface of the deformable mat -40- with multiple pressure sensors -43-, which are distributed uniformly within the mat, is advantageous, from which the total weight of the patient can be calculated. This is of advantage because from the volume and from the weight the density of the human body can be calculated and therefore directly also its fat and non fat components. Another design of the mat -40 could be a so called intelligent or smart mat, which is highly deformable, which recognises its own deformity and which fits itself consistently to the hindmost contours of the body which are not presentable. This mat could be produced e.g. from contacting foam (e.g. preferential conductivity in the depth direction of the mat), whereby through the deformation of the foam the electrical properties of the mat are changed; e.g. the resistance change in the sagittal direction induced by compression (in the depth of the mat) or changed capacities could be recorded or also the distension of the surface which arises from the compression of the mat at specific points. So e.g. a so called conducting foam is on the market produced e.g. from polyether-polyurethane and impregnated with structured carbon, whose resistance changes when it is compressed or elongated. The carbon is e.g. bound to e.g. the open foam structure via a synthetic polymeric latex. Also an inherently conducting material like Polypyrrol (Ppy) could be used for the coating of the e.g. open foam. An assembling of the mat from multiple small single elements of a conducting material, e.g. isolated from each other, is thought of.

One will choose of course a material with very good and reversible deformity. Possible changes of the electrical properties of the smart mat with changed temperature would have to be considered of course. It will be of advantage if the mat is isolated against the body and against the underlying structure with a thin coat of a highly electrically insulating material. It is also envisaged to use every method which will be developed in future or will be commercially available for this purpose. So a consistent and complete 3D-image of the surface of the body not being visible in the part where he lies will be delivered by the intelligent or smart mat as a so called negative phantom. In the visible part of the body the contactless 2D or 3D imaging of the body surface will be delivered. If the mat -40- has a defined elastic modulus, from the deformation also the weight of the patient can be derived without separate pressure sensors.

### LIST OF USED TERMS IN DRAWING

Instrument arm -1-
Examination table -2-
Contactless measuring device -3-
Examined body -4-
Electrodes 5a to 5d
Electrode brackets 6a to 6d
Swing device -7-
Measuring fields -8-
Cable -9-
Fs ECG -10p-
Thrust drive -11-
Measuring angle -12-
Table upper part -13-
Inclination angle -14-
Measurement trolley -15-
Display -16-
Rolls -17-
Swivel arm -18-
Joints -19-
Grid projector -20-
Ruler -21-
Double electrodes -22-
Change over switch -23-
Impedance meter -24-
Clamp -26-
Tension device -27-
Deepening -28.-
Carrier -29-
Suction electrode -30-
Pressure cuff -32-
Side wings -33-
Normal range field -34-
Reading point -35-
Trend line -36-
Triple electrode element -37-
Multi plug -38-
Distance provider -39-
Deformable mat -40-
Transverse rods -41-
Extending part of mat -42-
Pressure sensor -43-

1-73. (canceled)

74. A device for electronically measuring bodily functions, the device comprising:
a plurality of electrodes capable of being attached to a body;
a reference surface on which the body may be placed;
an electrical impedance meter capable of measuring the bodily functions of a two or three dimensional segment of the body by measuring the electrical impedance of the body and estimating the shape of the segment of the body by locating the electrodes attached to the body with reference to the reference surface on which the body is placed,
wherein the measured electrical impedance is associated with the estimated shape of the at least one body segment in order to measure the bodily functions of the at least one body segment.

75. The device according to claim 74, wherein the device comprises an electrocardiogram.

76. The device according to claim 74, wherein the shape of the segment of the body is estimated to be the shape of a cylinder or truncated cone.

77. The device according to claim 74, further comprising at least one projector capable of projecting a predetermined pattern onto the body.

78. The device according to claim 74, wherein the electrodes are capable of applying an alternating current of at least one frequency to the body in more than one direction, the electrical impedance meter being further capable of measuring the electrical impedance of the body segment in at least two different directions due to the alternating current over a period of time.

79. The device according to claim 78, wherein the electrodes comprise electrical relays or other electronic change-over switches which are capable of alternating the current.

80. The device according to claim 78, wherein the alternating current of at least one frequency is introduced into the body segment and that the resulting impedance, active resistance, reactance, or phase angle of the body segment in a predetermined period of time are measured.

81. The device according to claim 74, wherein the device is capable of measuring two body segments and measuring the distance between the two body segments.

82. The device according to claim 74, wherein at least five electrodes are attached to the body segment.

83. The device according to claim 74, wherein the device is capable of measuring the electrical and mechanical properties of the heart, the circulation through the body segment, the volume of the body segment, the change in volume of the body segment, or the composition of bodily fluids in the body segment.

84. The device according to claim 74, wherein the electrodes are attached to the body segment in groups of two or three electrodes.

85. The device according to claim 74, wherein the electrodes comprise electrocardiogram electrodes.

86. The device according to claim 74, further comprising a device arm for mounting the electrical impedance meter, the device arm comprising either a swivel arm, articulated arm, or a telescopic arm.

87. The device according to claim 74, wherein the electrodes are each labelled with a predetermined code and the device further comprises a mechanism capable of reading the predetermined codes of the electrodes.

88. The device according to claim 74, wherein the impedance is measured by transmitting at least two different frequencies of between 0.1 and 40 kHz, at least one frequency which is higher than 40 kHz, and at least one frequency that is smaller than 2 MHz.

89. The device according to claim 74, wherein the reference surface comprises a deformable mat comprised of transverse rods which are elastically interconnected in the longitudinal direction.

90. The device according to claim 74, wherein the reference surface further comprises pressure sensors which are capable of measuring the weight of a patient or of the body segments.

91. The device according to claim 74, further comprising a database capable of storing data from previous measurements that has been previously collected, wherein the measured bodily functions may be compared to data from previous measurements.

92. The device according to claim 74, further comprising a pressure increasing means which may be attached to the body segment in order to increase the pressure of the body segment, the increased pressure in the body segment being detectable by the device.
